(19) United States
(12) Patent Application Publication
(10) Pub. No.: US 2025/0190857 A1
(43) Pub. Date: Jun. 12, 2025

(54) EXPLORATORY DATA ANALYSIS AUTOMATION SYSTEM AND METHOD BASED ON VARIABLE ATTRIBUTES

(71) Applicant: YOOJINBIOSOFT CO., LTD., Goyang-si (KR)

(72) Inventors: Jin Tae YOU, Seoul (KR); Jin Ho YOO, Goyang-si (KR)

(21) Appl. No.: 18/547,454

(22) PCT Filed: Sep. 28, 2022
(86) PCT No.: PCT/KR2022/014606

§ 371 Date: Jan. 31, 2025

(30) Foreign Application Priority Data
Mar. 29, 2022 (KR) 10-2022-0038970

(51) Int. Cl. G06N 20/00

(52) U.S. Cl. CPC G06N 20/00

(57) ABSTRACT

Proposed is a data analysis automation system, and more particularly, an exploratory data analysis automation system based on variable attributes, the system enabling a data analysis to be automated considering variable attributes so that a data analysis is performed with an algorithm adaptively selected for a variety of generated variables.

### TECHNICAL FIELD

The present disclosure relates to a data analysis automation system. More particularly, the present disclosure relates to an exploratory data analysis automation system based on variable attributes, the system enabling a data analysis to be automated considering variable attributes so that a data analysis is performed with an algorithm adaptively selected for a variety of generated variables.

### BACKGROUND ART

In clinical fields, software equipped with various statistical analysis algorithms is frequently used to find factors that cause diseases or that are related to occurrence of diseases, and to analyze the effects of newly developed drugs or treatments.

Clinical researchers (doctors, nurses, and pharmacists) are required to write clinical theses in order to produce research results. When writing the clinical theses, clinical statistical analysis is required. It is very difficult for an individual clinical researcher with limited knowledge of statistical analysis to perform a clinical statistical analysis without expert help.

In particular, the terminologies used in medical specialties, such as gastroenterology/circulatory internal medicine, neurosurgery, orthopedics, etc., vary and consist of highly specialized words. Therefore, it is very difficult for an individual to perform a statistical analysis that requires long learning without expert help. Since the experience of a researcher who has produced data is very important for data analysis, it is difficult for even a statistician having no clinical knowledge to suitably perform a clinical statistical analysis alone without the help of a clinical researcher.

Therefore, there is a need for a software program that facilitates a statistical analysis suitable for clinical researchers who understands the characteristics of data and research objectives well.

Conventional statistical analysis software is difficult to use because statistical algorithms and parameters are wildly scattered. This makes interpretation of analysis results difficult and requires a significant amount of time for manual extraction/editing of the results, which may also lead to errors in editing.

Therefore, it is very difficult for a non-statistician to use the conventional statistical analysis software and properly produce a result of analysis that the researcher wants.

The applicant invented and proposed Korean Patent Application No. 10-2011-0104734, “METHOD OF AUTOMATICALLY EXPLORING ASSOCIATIONS BETWEEN VARIABLES AND GENERATING DYNAMIC RESULT REPORT BY USING SAME”.

According to the method of automatically exploring associations between variables and generating dynamic result report by using same, the characteristics of a number of clinical and epidemiological variables used in medical statistics are understood for automatic classification into most commonly used types. In addition, when there are a large number of variables to be analyzed and of statistical algorithms to be applied, associations between all the variables are automatically specified, and a comprehensive result report enabling an at-a-glance view of the numerous associations analyzed between the clinical and epidemiological variables is provided.

In this conventional invention, most analysis parameters frequently used in a statistical analysis are predetermined. In addition, the conventional invention is based on the following idea: a statistical algorithm is automatically selected according to the variable characteristics, even for a complicated analysis performed by statisticians, considering the process of the analysis in details, wherein the process is converted into a process of selecting two or more conditions, which may be provided as propositions, through several steps.

The invention of the applicant has technical features that the characteristics of a number of clinical and epidemiological variables used in medical statistics are understood for automatic classification into the most frequently used type and when there are many variables to be analyzed and statistical algorithms to be applied, every association between all the variables is automatically designated.

The conventional method of automatically exploring associations between variables is to separate types of clinical and epidemiological variables considering the characteristics thereof according to a process determined in a statistical algorithm and to determine a statistical algorithm to be automatically applied, accordingly. A statistical algorithm is automatically applied according to the variable characteristics, so that even users who do not know statistical analysis methods or particular statistical algorithms well may easily use a statistical analysis program.

In recent years, clinical processes and fields have been further diversely segmented, and in clinical sites, such as hospitals in clinical phase, several tens to hundreds of pieces of variable data have been produced depending on the field. Therefore, it is difficult to specify relations between all variables, and there is a limitation in selecting appropriate statistical algorithms by clinical researchers, most of whom are non-statisticians.

### DISCLOSURE

### Technical Problem

The present disclosure is intended to propose a data analysis automation system as an extension of the related art “METHOD OF AUTOMATICALLY EXPLORING ASSOCIATIONS BETWEEN VARIABLES AND GENERATING DYNAMIC RESULT REPORT BY USING SAME”, and is intended to propose the data analysis automation system adaptive for a variety of generated variables. The present disclosure is directed to providing an exploratory data analysis automation system based on variable attributes, the system being capable of understanding the characteristics of the variables automatically and selecting an appropriate statistical algorithm and obtaining an analysis result automatically.

Taking this into account, the present disclosure is directed to providing an exploratory data analysis automation system based on variable attributes, wherein pre-screening for a number of variables is provided and an optimal data analysis algorithm based on data characteristics (variable characteristics) is automatically applied, so that on the basis of clinical researchers' data characteristics (variable characteristics), data analysis is performed well to meet the research objectives.

### Technical Solution

Clinical research objectives are often expressed in a concise sentence, and implicit clinical research objectives are often expressed in a concise, implicit sentence (e.g., “an analysis of cytokine change difference between different treatment groups”), to achieve the research objectives,

(a). a normal distribution test, (b). a test for equality of variance between groups, (c). a post-test, and (d). determining a nonparametric method used when a normal distribution is not followed need to be performed, which are difficult for clinical researchers, most of whom are non-statisticians, to perform on their own.

Therefore, there is a great need for software that automatically selects an optimal statistical algorithm for clinical researchers on the basis of the characteristics (variable characteristics) of their data to best fulfill their research objectives.

Thus, the present disclosure is directed to providing an exploratory data analysis automation system based on variable attributes, the system capable of automatically selecting a statistical algorithm optimized for achieving research objectives.

According to the present disclosure, an exploratory data analysis automation system based on variable attributes includes:
a variable attribute definition means for extracting features of variables included in data, and classifying the variables according to forms and features of constituent values of the variables, and defining attributes of the classified variables; an algorithm management means for storing and managing data analysis algorithms and providing the algorithms according to a request of a data analysis control means; the data analysis control means for defining attributes of variables included in data through the variable attribute definition means, and selecting an algorithm for a data analysis according to the attributes of the variables, and controlling execution of the data analysis of a data analysis means; and the data analysis means for performing a data analysis according to an algorithm set by the data analysis control means and providing result information as a distribution analysis result table and a distribution analysis result figure,
wherein the variable attribute definition means is configured to perform classification into first classification variables and second classification variables and define attributes thereof, wherein the first classification variables are classified into continuous variables and categorical variables according to forms of constituent values of variables included in data and the categorical variables are classified into ordinal variables and nominal variables, and the second classification variables include single-measured variables, repeatedly measured variables, and survival data according to features of variables included in data, and
the data analysis control means includes: a variable relation analysis control means for performing a variable relation analysis of automatically classified variables according to a combination of types of the variables with reference to variable attributes to determine whether a single-variable distribution analysis or an analysis of a relation between two or more variables is performed; a single-variable distribution analysis control means for performing data analysis control for each continuous variable or each categorical variable when it is determined that a single-variable distribution analysis is performed according to a variable relation analysis result of the variable relation analysis control means; and a variable-to-variable relation analysis control means for setting an algorithm and performing data analysis control by distinguishing between cases of all single-measured variables, all repeatedly measured variables, a mix of single-measured and repeatedly measured variables, and survival data when it is determined that an analysis of a relation between two or more variables is performed according to a variable relation analysis result of the variable relation analysis control means.

According to the present disclosure, an exploratory data analysis automation method based on variable attributes includes:
a variable attribute definition process of defining variable attributes for data analysis automation; and selecting and setting an algorithm for a data analysis according to the variable attributes,
wherein the variable attribute definition process for defining one variable attribute includes
performing classification into first classification variables and second classification variables and defining attributes thereof, wherein the first classification variables are classified into continuous variables and categorical variables according to forms of constituent values of variables included in data and the categorical variables are classified into ordinal variables and nominal variables, and the second classification variables include single-measured variables, repeatedly measured variables, and survival data according to features of variables included in data, and
a data analysis process of selecting an algorithm for a data analysis according to variable attributes and performing the data analysis includes the following processes:
a variable relation analysis process in which a variable relation analysis of variables automatically classified is performed according to a combination of types of the variables and it is determined whether a single-variable distribution analysis or an analysis of a relation between two or more variables is performed,
a single-variable distribution analysis process in which according to a variable relation analysis result of the variable relation analysis process, in the case of the single-variable distribution analysis, distribution for each continuous variable or categorical variable is analyzed and a result is provided as a distribution analysis result table and a distribution analysis result figure, and
a variable-to-variable relation analysis process in which according to a variable relation analysis result of the variable relation analysis process, in the case of the analysis of a relation between two or more variables, an algorithm is selected and set by distinguishing between cases of all single-measured variables, all repeatedly measured variables, a mix of single-measured and repeatedly measured variables, and survival data, and a data analysis result is provided.

### Advantageous Effects

According to the present disclosure, a data analysis is performed by selecting an optimal algorithm based on variable attributes, thereby providing a data analysis automation system adaptive for variable data that are more diversely increased as clinical processes and fields are diversely segmented.

### DESCRIPTION OF DRAWINGS

FIG. 1 is a block diagram illustrating a configuration of an exploratory data analysis automation system based on variable attributes according to the present disclosure.

FIG. 2 is a flowchart illustrating a data analysis control process in the present disclosure.

FIG. 3 is a flowchart illustrating a data analysis control process for continuous variable distribution in the present disclosure.

FIG. 4 is a flowchart illustrating a data analysis control process for categorical variable distribution in the present disclosure.

FIG. 5A, FIG. 5B, and FIG. 5C are diagrams illustrating a result of a continuous-variable data analysis in the present disclosure, wherein FIG. 5a is a histogram, FIG. 5b is a Q-Q plot, and FIG. 5c is a box plot.

FIG. 6 is a bar plot illustrating a result of a data analysis of categorical variable distribution in the present disclosure.

FIG. 7 is a flowchart illustrating a process of analyzing a continuous variable-to-continuous variable relation in the present disclosure.

FIG. 8 is a flowchart illustrating a normal distribution test process for continuous variables in the present disclosure.

FIG. 9 is a scatter plot (correlation scatter plot) illustrating a result of analyzing a correlation between continuous variables in the present disclosure.

FIG. 10 is a flowchart illustrating a data analysis control process for a continuous variable-to-categorical variable relation in the present disclosure.

FIG. 11 is a flowchart illustrating an analysis control process for a categorical variable-to-categorical variable relation in the system according to the present disclosure.

FIG. 12 is a flowchart illustrating a data analysis control process for a relation between three or more variables that are all single-measured in the present disclosure.

FIG. 13 is a flowchart illustrating a data analysis control process for a case in which all variables are repeatedly measured in the present disclosure.

FIG. 14 is a flowchart illustrating a data analysis control process for a case in which a single-measured variable is mixed in the present disclosure.

FIG. 15 is a flowchart illustrating a data analysis control process for survival data in the present disclosure.

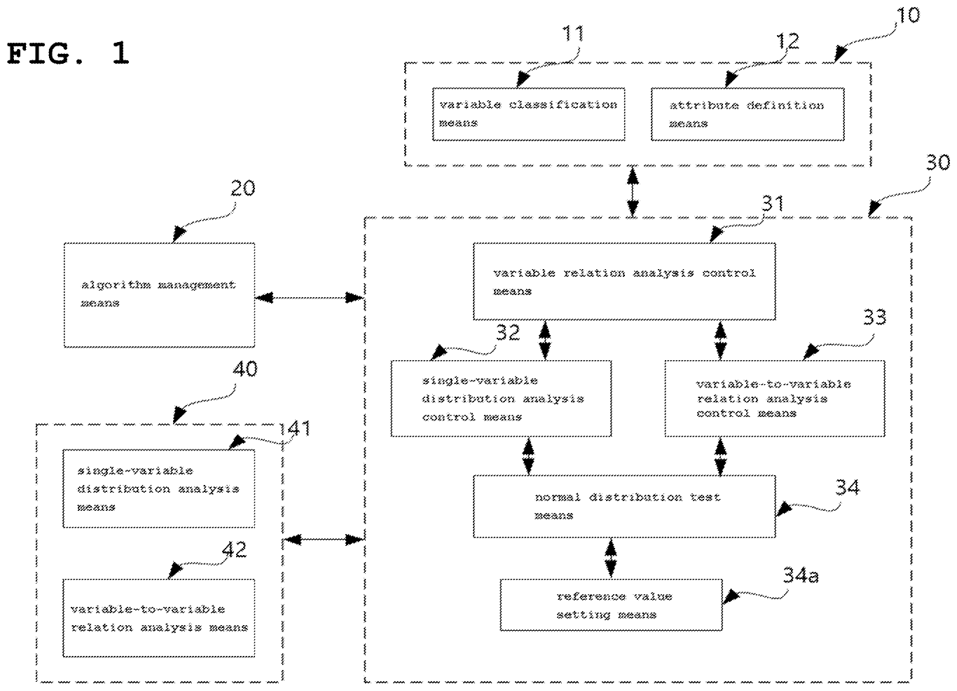

### BEST MODE

According to the present disclosure, an exploratory data analysis automation system based on variable attributes includes:
a variable attribute definition means 10 for defining attributes of variables for data analysis automation; an algorithm management means 20 for storing and managing data analysis algorithms and providing the algorithms according to a request of a data analysis control means 30; the data analysis control means 30 for defining attributes of variables included in data through the variable attribute definition means 10, and selecting an algorithm for a data analysis according to the variable attributes, and controlling the execution of the data analysis of a data analysis means 40; and the data analysis means 40 for performing a data analysis according to an algorithm set by the data analysis control means 30 and providing result information.

The variable attribute definition means 10 includes: a variable classification means 11 for extracting features of variables included in data and classifying the variables according to the forms and features of constituent values of the variables; and an attribute definition means 12 for defining attributes of the variables classified by the variable classification means 11.

The variable classification means 11 performs classification into first classification variables and second classification variables. The first classification variables include continuous variables and categorical variables according to forms of constituent values of variables included in data. The second classification variables include single-measured variables, repeatedly measured variables, and survival data according to features of variables included in data.

The categorical variables may be classified into an ordinal variable and a nominal variable.

The continuous variables are variables that represent age, body mass index, height, etc. The ordinal variable is a categorical variable having order, such as an age of 40 or older, or an age younger than 40. The nominal variable is a categorical variable not having order, such as a male/female variable.

The single-measured variables means variables (e.g., a health checkup value measured in 2022) representing values measured one time and independently. The repeatedly measured variables mean variables (e.g., a hemoglobin value measured before surgery/i month after surgery/i year after surgery) representing values measured repeatedly for a predetermined period. The survival data means variables (e.g., survival/censored/death information for 10 years of follow-up) representing sample status and duration information.

The data analysis control means 30 includes: a variable relation analysis control means 31, a single-variable distribution analysis control means 32, and a variable-to-variable relation analysis control means 33. The variable relation analysis control means 31 performs a variable relation analysis of automatically classified variables according to a combination of the types of the variables with reference to variable attributes to determine whether a single-variable distribution analysis or an analysis of a relation between two or more variables is performed. In the case of a single-variable distribution analysis according to a variable relation analysis result of the variable relation analysis control means 31, the single-variable distribution analysis control means 32 controls an analysis of data for each continuous variable or each categorical variable. In the case of an analysis of a relation between two or more variables according to a variable relation analysis result of the variable relation analysis control means 31, the variable-to-variable relation analysis control means 33 sets an algorithm and controls an analysis of data by distinguishing between the cases of all single-measured variables, all repeatedly measured variables, a mix of single-measured and repeatedly measured variables, and survival data.

The data analysis means 40 includes a single-variable distribution analysis means 41, and a variable-to-variable relation analysis means 42. According to the control of the single-variable distribution analysis control means 32, the single-variable distribution analysis means 41 analyzes data for each continuous variable or each categorical variable and provides a result as a distribution analysis result table and a distribution analysis result figure. According to the control of the variable-to-variable relation analysis control means 33, the variable-to-variable relation analysis means 42 analyzes data and provides a result by performing an algorithm set depending on a relation between variables for the cases of all single-measured variables, all repeatedly measured variables, a mix of single-measured and repeatedly measured variables, and survival data.

In addition, the data analysis control means 30 includes a normal distribution test means 34 for a continuous variable. The normal distribution test means 34 calculates a significance probability value (p value; p1) through execution of Lilliefors test for a continuous variable, calculates a significance probability value (p value: p2) through execution of Shapiro-Wilk test, and produces a normal distribution test result for the continuous variable through comparison of a reference value (α) with p1 and p2.

According to the normal distribution test result for the continuous variable, when the condition p1<α (AND or OR) p2<α is satisfied, it is determined that a normal distribution is not followed, or when the condition is not satisfied, it is determined that the normal distribution is followed.

The normal distribution test means 34 for a continuous variable may include a reference value setting means 34a that provides a reference value setting process to allow a user to set the reference value (α).

The single-variable distribution analysis means 41 may provide, as a result of a data analysis of a continuous variable, continuous variable distribution in the form of a distribution analysis result table, a histogram, a Q-Q plot, and a box plot, and may provide categorical variable distribution in the form of a distribution analysis result table and a bar plot as a data analysis result.

Values calculated from the continuous variable distribution analysis result table include:
(a). Total N: the total number of samples included in data
(b). Valid N (%) the number and % of samples excluding missing values
(c). Missing N (%) the number and % of missing samples
(d). Min˜Max the minimum maximum value of the variable
(e). Mean±standard deviation the mean±standard deviation of the variable
(f). Mean (95% CIs) the mean (95% confidence interval) of the variable
(g). Median (IQR) the median (inter-quartile range) of the variable
(h). Skewness the skewness of the variable
(i). Kurtosis the kurtosis of the variable
(j). Lilliefors test for normality, p value
   : a significance probability value (p value) resulting from testing the normal distribution of the variable with the Lilliefors method
(k). Shapiro-Wilk test for normality, p value: a p value resulting from testing the normal distribution of the variable with the Shapiro-Wilk method

Values calculated from the categorical variable distribution analysis result table include:
(a). Total N: the total number of samples included in data
(b). Valid N (%): the number and % of samples excluding missing values
(c). Missing N (%) the number and % of missing samples
(d). Subgroup: the name of a subgroup included in the categorical variable
(e). N (%) the number and % of samples included in the subgroup
(f). 95% CI the 95% confidence interval of the number % of samples included in the subgroup

The variable-to-variable relation analysis control means 33
provides a process of setting an algorithm and controlling an analysis of data by distinguishing between the cases of all single-measured variables, all repeatedly measured variables, a mix of single-measured and repeatedly measured variables, and survival data.

### [When all Variables are Single-Measured]

When all variables are single-measured, a data analysis is performed by distinguishing between an analysis of a relation between two variables and an analysis of a relation between three or more variables. The following process is included. In the case of an analysis of a relation between two variables, a data analysis is performed according to a continuous variable-to-continuous variable relation, a continuous variable-to-categorical variable relation, and a categorical variable-to-categorical variable relation. In the case of an analysis of a relation between three or more variables, a data analysis is performed by distinguishing between the cases of all continuous variables, all categorical variables, and a mix of continuous and categorical variables.

### [A Continuous Variable-to-Continuous Variable Relation]

In the case of the continuous variable-to-continuous variable relation, a data analysis control process includes: performing a normal distribution test for two continuous variables; and determining whether all the two variables follow a normal distribution as a normal distribution test result, and setting Pearson correlation analysis to perform data analysis control when the normal distribution is followed or setting Spearman correlation analysis to perform data analysis control when the normal distribution is not followed, and providing an analysis result in the form of an analysis result table and an analysis result figure.

Values provided in an analysis result table as an analysis result for the continuous variable-to-continuous variable relation include:
(a). A correlation coefficient
(b). The 95% confidence interval of a correlation coefficient
(c). A significance probability value (p value) calculated as a result of testing correlation coefficient=0

An analysis result figure provided as an analysis result for the continuous variable-to-continuous variable relation may be a correlation scatter plot in which x-axis and y-axis variables are set and a regression curve is represented.

### [A Continuous Variable-to-Categorical Variable Relation]

In the case of the continuous variable-to-categorical variable relation, a data analysis control process includes:
performing a data analysis by distinguishing between a mean difference analysis, a correlation analysis, and a binary response prediction performance analysis.

The mean difference analysis includes: extracting the number (m) of categorical variable subgroups and performing a normal distribution test for a continuous variable when the extracted number (m) of categorical variable subgroups is two or when the extracted number of categorical variable subgroups is three or more; determining whether the continuous variable follows a normal distribution when the number (m) of categorical variable subgroups is two; setting Wilcoxon rank-sum test when the number (m) of categorical variable subgroups is two and the continuous variable does not follow the normal distribution, or performing Levene's test when the number (m) of categorical variable subgroups is two and the continuous variable follows the normal distribution to test whether the variances of the subgroups are the same, and setting “Student's Test” when the variances of the subgroups are the same or setting “Welch's T test” when the variances of the subgroups are not the same to perform data analysis control; or setting 1-way ANOVA to perform data analysis control when the number (m) of categorical variable subgroups is three or more and the continuous variable follows a normal distribution, or setting Kruskal-Wallis H test to perform data analysis control when the continuous variable does not follow the normal distribution.

In addition, in the case of the continuous variable-to-categorical variable relation, the data analysis control process further includes
performing and controlling post-validation (post-hoc analysis) after the data analysis with the 1-way ANOVA or the Kruskal-Wallis H test.

Algorithms used in post-validation performed after the data analysis with the 1-way ANOVA include Bonferroni test, Tukey test, Scheffe test, and Dunnett test. Algorithms used in post-validation after the data analysis with the Kruskal-Wallis H test include Bonferroni test, FDR (False Discovery rate), and Dunn's test.

In addition, the binary response prediction performance analysis includes: extracting the number (m) of categorical variable subgroups; and setting a ROC curve analysis to perform data analysis control when the extracted number (m) of categorical variable subgroups is two.

The correlation analysis includes: performing a normal distribution analysis for a continuous variable; determining whether the continuous variable follows a normal distribution; determining whether the categorical variable is an ordinal when the continuous variable follows the normal distribution; setting Polyserial correlation analysis to perform data analysis control when the categorical variable is an ordinal or setting Point polyserial correlation analysis to perform data analysis control when the categorical variable is not an ordinal; or determining whether the categorical variable is an ordinal variable when the continuous variable does not follow the normal distribution; and setting Polychoric correlation analysis to perform data analysis control when the categorical variable is an ordinal variable or setting Rank polyserial correlation analysis to perform data analysis control when the categorical variable is not an ordinal variable.

### [A Categorical Variable-to-Categorical Variable Relation]

In the case of the categorical variable-to-categorical variable relation, a data analysis control process includes:
distinguishing between a case in which the number (m,n) of subgroups included in each categorical variable is two and other cases, and performing data analysis control with an independence test when the number (m,n) of subgroups included in each categorical variable is two or by distinguishing between an independence test, a trend test, and a correlation analysis when the number (m,n) of subgroups included in each categorical variable is not two.

The independence test includes: constructing a 2×2 cross-table of subgroups for two categorical variables, constructing an expected-value table assuming that the categorical variables are independent, and determining whether the number of cells with an expected value<5 in an m×n cross-table for the number (m,n) of subgroups included in each categorical variable is equal to or greater than 25% of the total number of cells; and considering computer computational capacity when the number of cells with an expected value<5 in the m×n cross-table for the number (m,n) of subgroups included in each categorical variable is equal to or greater than 25% of the total number of cells, and setting Fisher's exact test when the computer computational capacity is sufficient or setting Chi-squared test with Yates correction when the computer computational capacity is insufficient, or determining whether the cross-table is in a 2×2 form when the number of cells with an expected value<5 in the m×n cross-table for the number (m,n) of subgroups included in each categorical variable is less than 25% of the total number of cells, and setting Chi-squared test with Yates correction when the cross-table is in a 2×2 form or setting Chi-squared test when the cross-table is not in a 2×2 form to perform data analysis control.

The trend test includes: constructing an m×n cross-table of subgroups of two categorical variables and determining whether the condition m≥3 and n≥3 is satisfied; and selecting and setting Linear by linear association test when the condition m≥3 and n≥3 is satisfied or setting Cochran'Qtest when the condition m≥3 and n≥3 is not satisfied to perform data analysis control.

The correlation analysis includes: determining whether all the categorical variables are ordinals, selecting and setting Polychoric correlation analysis when all the categorical variables are ordinals or determining whether all the categorical variables are nominal variables when not all the categorical variables are ordinals, and selecting and setting Cramer's V analysis when all the categorical variables are nominal variables or setting Rank polyserial correlation analysis when not all the categorical variables are nominal variables to perform data analysis control.

[An Analysis of a Relation Between Three or More Variables that are all Single-Measured]

For the analysis of a relation between three or more variables that are all single-measured, a data analysis control process

performs a data analysis by distinguishing between the cases in which three or more variable groups are all continuous, are all categorical, and continuous and categorical variables are mixed.

When the three or more variable groups are all continuous, PCA (Principal Component Analysis) is set to perform data analysis control.

When continuous and categorical variables are mixed, the following procedures are performed: using each continuous variable as a dependent variable and setting univariable linear regression to analyze individual influence of the remaining variables on the continuous variable; using each continuous variable as a dependent variable and setting multivariable linear regression to analyze combined influence of the remaining variables on the continuous variable; using each continuous variable as a dependent variable and setting ANCOVA (Analysis of covariance) to perform data analysis control when the remaining variables include a categorical variable, and using each continuous variable as a dependent variable and performing 2-way ANOVA to perform data analysis control when the remaining variables are all categorical variables.

When all variables are categorical variables, the following procedures are performed: determining whether there are binary variables; using each binary variable as a dependent variable when there are binary variables and setting univariable binary logistic regression to analyze individual influence of the remaining variables on the binary variables, and setting multivariable binary logistic regression to analyze combined influence of the remaining variables on the binary variables; building a binary response prediction model; and performing validation analysis control for the built binary response prediction model.

When there are no binary variables and all ternary or higher variables, the following procedures are performed: using each variable as a dependent variable, and assuming the ternary variables are ordinal variables or nominal variables, and analyzing individual influence and combined influence of the remaining variables on the ternary ordinal variables or the ternary nominal variables, wherein univariable ordinal logistic regression is set to analyze the individual influence of the remaining variables on the ternary ordinal variables, multivariable ordinal logistic regression is set to analyze the combined influence of the remaining variables on the ternary ordinal variables, univariable nominal logistic is set to analyze the individual influence of the remaining variables on the ternary nominal variables, and multivariable nominal logistic regression is set to analyze the combined influence of the remaining variables on the ternary nominal variables.

In addition, a validation analysis for the binary response prediction model is performed through a discrimination aspect prediction performance analysis, a calibration aspect prediction performance analysis, and a model performance cross-validation analysis.

Indexes used in the discrimination aspect prediction performance analysis include:
(a). Performance analysis indexes (including the 95% confidence interval)
   AUC (95% CI)
   Sensitivity, Specificity
   PPV (positive predictive value), NPV (negative predictive value)
   ACC (accuracy), MIS (miss-classification rate)
   FPR (False Positive Rate), FNR (False Negative Rate), FDR (False Discovery Rate), FOR (False Omission Rate)
   LR+ (Positive Likelihood Ratio), LR− (Negative Likelihood Ratio), DOR (Diagnostic Odds Ratio)
(b). Visualization of a performance analysis result
   ROC curve

Indexes used in the calibration aspect performance analysis include:
(a). Performance analysis indexes
   AIC (Akaike Information Criterion)
   BIC (Bayes Information Criterion)
   Nagelkerke R2
   Hosmer-Lemeshow test P value
   Brier score
   Spigelhalter Z score with P value
   Linear regression line in Calibration plot
   Intercept, 95% confidence interval, and p value
   Slope, 95% confidence interval, and p value
(b). Visualization of a performance analysis result
   Calibration plot
   Decile plot
   Calibration belt

The model performance cross-validation analysis includes:
(a). Methods used in performance cross-validation
   LOOCV (leave-one-out cross-validation)
   K-fold CV (cross validation)
   Permutation test
   Bootstrapping
(b). Visualization of a cross-validation result
   ROC curve
   Calibration plot
   Decile plot
   Calibration belt
      [An Analysis when all Variables are Repeatedly Measured]

For the analysis when all variables are repeatedly measured, a data analysis control process

performs a data analysis by distinguishing between the cases in which there are two repeatedly measured variables and there are three or more repeatedly measured variables, and between the cases in which repeatedly measured variables are continuous variables and repeatedly measured variables are categorical variables.

When there are two repeatedly measured variables and the repeatedly measured variables are continuous variables, a normal distribution analysis for the continuous variables is performed to determine whether the continuous variables follow a normal distribution. Paired sample T test is set when the normal distribution is followed or Wilcoxon signed-rank test is set when the normal distribution is not followed to perform data analysis control.

In addition, the data analysis control process may further include, when there are two repeatedly measured variables and the repeatedly measured variables are continuous variables, setting ICC (Intraclass Correlation Coefficient) analysis to perform data analysis control.

When there are two repeatedly measured variables and the repeatedly measured variables are categorical variables, the following procedures are performed. When the number (m,n) of subgroups included in each categorical variable is two (m=2, n=2), McNemar's test and Cohens'Kappa are set to perform data analysis control. When the number (n) of subgroups included in one categorical variable is two and the number (n) of subgroups included in the other is three or more (m=2, n≥3), Cochran-Armitage test for trend is set to perform data analysis control. When the number (m,n) of subgroups included in each categorical variable is three or more (m≥3,n≥3), McNemar-Bowker test is set to perform data analysis control.

When there are three or more repeatedly measured variables and the repeatedly measured variables are continuous variables, Linear mixed effect model analysis, GEE (Generalized Estimating Equation) analysis, and Repeated measures 1-way ANOVA are set to perform data analysis control.

When there are three or more repeatedly measured variables and the repeatedly measured variables are categorical variables, Generalized mixed effect model analysis and GEE (Generalized Estimating Equation) analysis are set to perform data analysis control.

[An Analysis when a Single-Measured Variable is Mixed]

When a single-measured variable is mixed, in a data analysis control process,

A case in which a repeatedly measured variable is a continuous variable and a case in which a repeatedly measured variable is a categorical variable are distinguished.

When a repeatedly measured variable is a continuous variable, Linear mixed effect model analysis and GEE (Generalized Estimating Equation) analysis are set to perform data analysis control. When a repeatedly measured variable is a continuous variable and a single-measured variable is a categorical variable, Repeated measures 2-way ANOVA is set to perform data analysis control.

When a repeatedly measured variable is a categorical variable, Generalized mixed effect model analysis, and GEE (Generalized Estimating Equation) analysis are set to perform data analysis control.

### [Survival Data Analysis]

In a data analysis control process for the survival data,
a case in which there are only survival time and an event occurrence variable and a case in which there are survival time, an event occurrence variable, and single-measured data are distinguished.

When there are only survival time and an event occurrence variable, Kaplan-Meier curve analysis is set to perform data analysis control. When there are survival time, event occurrence data, and single-measured data, univariable cox proportional hazards regression is set to control an analysis of individual influence of the single-measured variables on survival, multivariable cox proportional hazards regression is set to control an analysis of combined influence of the single-measured variables on survival, and Kaplan-Meier curve analysis is set to control Log rank test (comparison of differences in survival probability between subcategories).

In addition, the data analysis control process for the survival data
may further include: building a survival probability prediction model; and performing a discrimination aspect prediction performance analysis at Time=t, a calibration aspect prediction performance analysis at time=t, and a survival probability prediction model cross-validation analysis to analyze and control the survival probability prediction model.

Indexes used in the discrimination aspect prediction performance analysis at Time=t include, as described above:
(a). Performance analysis indexes (including the 95% confidence interval)
   AUC (95% CI)
   Sensitivity, Specificity
   PPV (positive predictive value), NPV (negative predictive value)
   ACC (accuracy), MIS (miss-classification rate)
   FPR (False Positive Rate), FNR (False Negative Rate), FDR (False Discovery Rate), FOR (False Omission Rate)
   LR+ (Positive Likelihood Ratio), LR− (Negative Likelihood Ratio), DOR (Diagnostic Odds Ratio)
(b). Visualization of a performance analysis result
   ROC curve

Indexes used in the calibration aspect performance analysis at Time=t include:
(a). Performance analysis indexes
   AIC (Akaike Information Criterion)
   BIC (Bayes Information Criterion)
   Nagelkerke R2
   Hosmer-Lemeshow test P value
   Brier score
   Spigelhalter Z score with P value
   Linear regression line in Calibration plot
   Intercept, 95% confidence interval, and p value
   Slope, 95% confidence interval, and p value
(b). Visualization of a performance analysis result
   Calibration plot
   Decile plot
   Calibration belt

The survival probability prediction model performance cross-validation at time=t includes:
(a). Methods used in performance cross-validation
   LOOCV (leave-one-out cross-validation)
   K-fold CV (cross validation)
   Permutation test
   Bootstrapping
(b). Visualization of a cross-validation result
   ROC curve
   Calibration plot
   Decile plot
   Calibration belt

A data analysis automation operation process of the exploratory data analysis automation system based on variable attributes according to the present disclosure will be described.

For data analysis automation, the variable attribute definition means 10 defines attributes of variables included in data.

The data analysis control means 30 classifies the variables included in the data according to the attributes of the variables through the variable attribute definition means 10 and controls a data analysis accordingly. According to the attributes of the variable, the data analysis control means 30 selects an algorithm to be used in the data analysis by the data analysis means 40 from the algorithm management means 20 and sets the algorithm to perform data analysis control.

The variable classification means 11 of the variable attribute definition means 10 extracts features of the variables included in data and classifies the variables according to the forms and features of constituent values of the variables.

The variable classification means 11 performs classification into first classification variables and second classification variables. The first classification variables include continuous variables and categorical variables according to forms of constituent values of variables included in data. The second classification variables include single-measured variables, repeatedly measured variables, and survival data according to features of variables included in data.

Herein, the categorical variables may be classified into an ordinal variable and a nominal variable.

The continuous variables are variables that represent age, body mass index, height, etc. The ordinal variable is a categorical variable having order, such as an age of 40 or older, or an age younger than 40. The nominal variable is a categorical variable not having order, such as a male/female variable.

The single-measured variables means variables (e.g., a health checkup value measured in 2022) representing values measured one time and independently. The repeatedly measured variables mean variables (e.g., a hemoglobin value measured before surgery/i month after surgery/i year after surgery) representing values measured repeatedly for a predetermined period. The survival data means variables (e.g., survival/censored/death information for 10 years of follow-up) representing sample status and duration information.

The data analysis control means 30 performs a variable relation analysis of automatically classified variables according to a combination of the types of the variables with reference to variable attributes, and analyzes data according to a result of the variable relation analysis. Herein, it may be determined whether a single-variable distribution analysis or an analysis of a relation between two or more variables is performed.

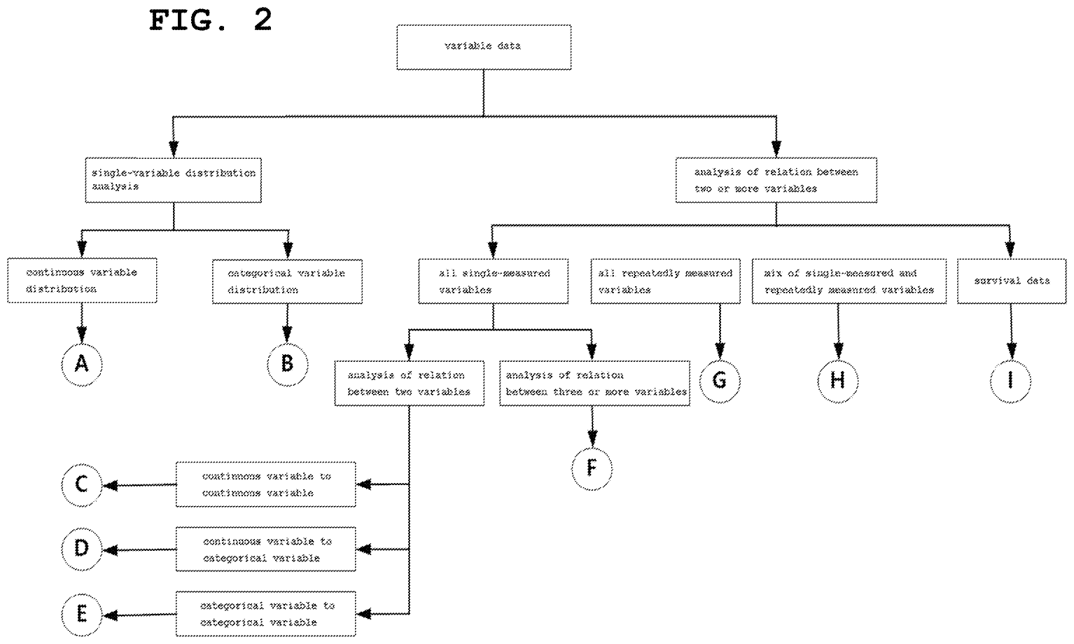

FIG. 2 is a flowchart illustrating a process of analyzing data according to variable attributes for variable data in the system according to the present disclosure.

A single-variable distribution analysis for variable data is a single-variable analysis for (A). continuous variable distribution and (B). categorical variable distribution, and for an analysis of a relation between two or more variables, a data analysis is performed according to the relation between the variables.

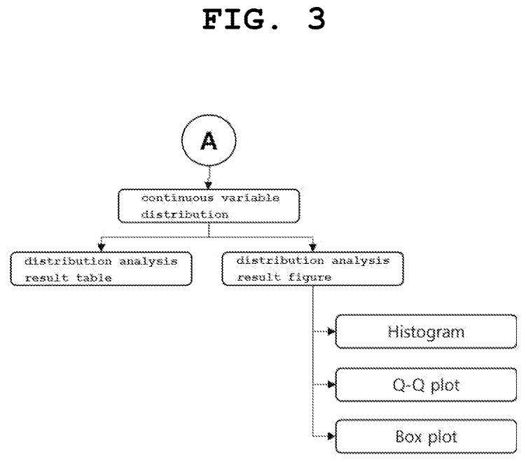

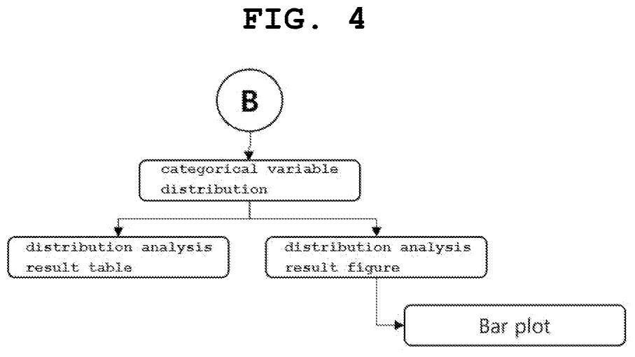

First, in the case of the single-variable distribution analysis, as shown in FIGS. 3 and 4, data is analyzed for each continuous variable or each categorical variable, and a result is provided as a distribution analysis result table and a distribution analysis result is provided as a figure.

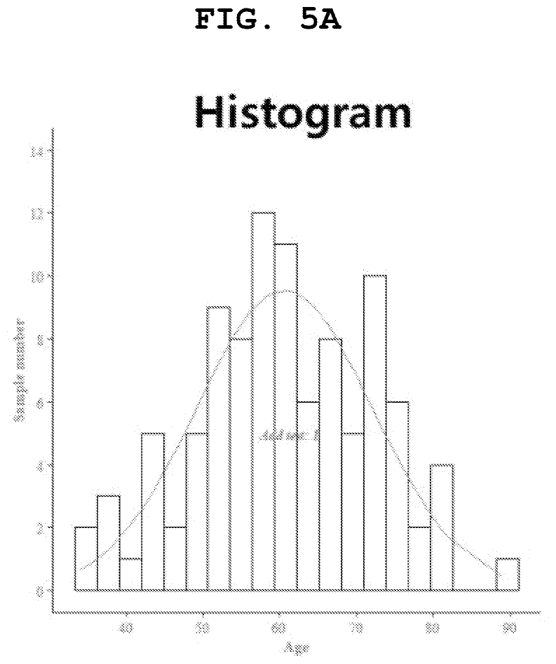

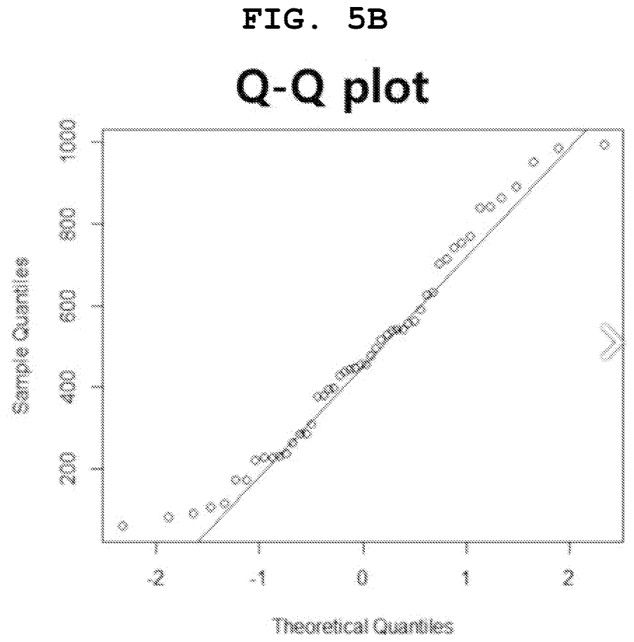

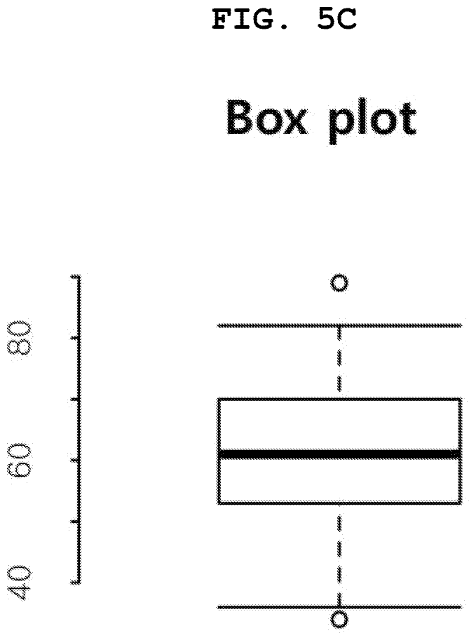

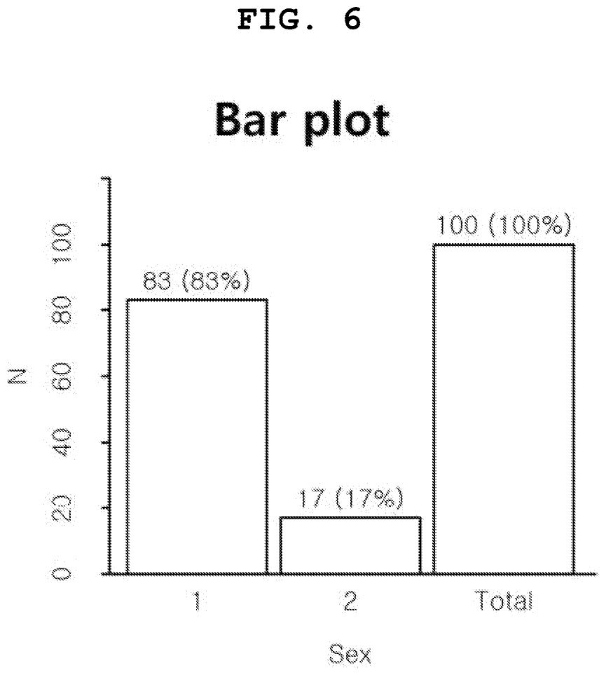

The single-variable distribution analysis means 41 may provide, as a result of a data analysis of a continuous variable, continuous variable distribution in the form of a distribution analysis result table, a histogram, a Q-Q plot, and a box plot as shown in FIGS. 5a, 5b, and 5c, and may provide categorical variable distribution in the form of a distribution analysis result table and a bar plot, as shown in FIG. 6, as a data analysis result.

In the meantime, for an analysis of a relation between two or more variables, an algorithm may be set and a data analysis is performed by distinguishing between the cases of all single-measured variables, all repeatedly measured variables, a mix of single-measured and repeatedly measured variables, and survival data.

### [When all Variables are Single-Measured]

When all variables are single-measured, a data analysis is performed by distinguishing between an analysis of a relation between two variables and an analysis of a relation between three or more variables.

The analysis of a relation between two variables includes a process in which a data analysis is performed according to (C). a continuous variable-to-continuous variable relation, (D). a continuous variable-to-categorical variable relation, and (E). a categorical variable-to-categorical variable relation, and for (F). an analysis of a relation between three or more variables, a data analysis is performed by distinguishing between the cases of all continuous variables, all categorical variables, and a mix of one continuous variable and one categorical variable.

### [A Continuous Variable-to-Continuous Variable Relation]

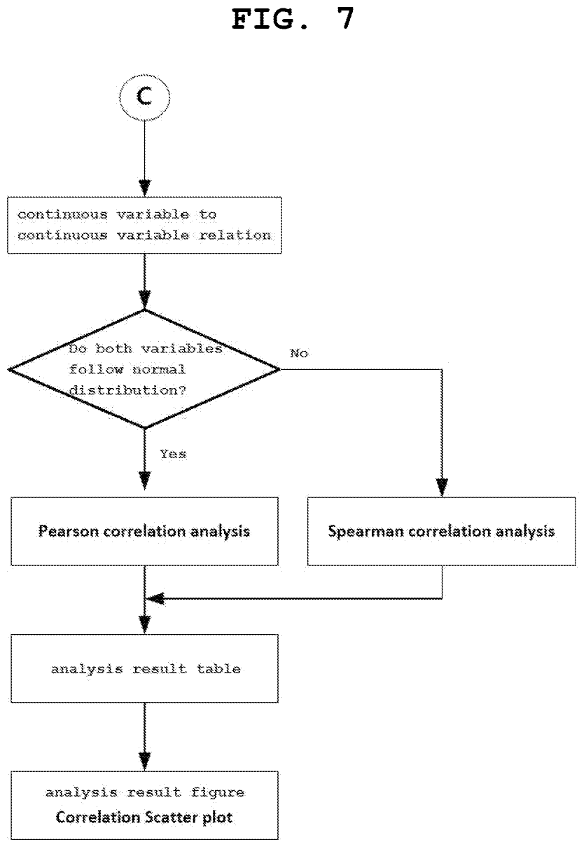

FIG. 7 is a flowchart illustrating a process of analyzing a continuous variable-to-continuous variable relation in the system according to the present disclosure.

In the case of the continuous variable-to-continuous variable relation, a normal distribution test for two continuous variables is performed.

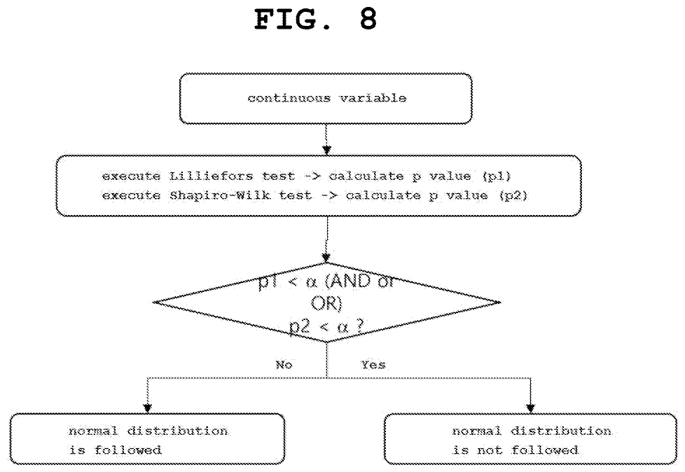

FIG. 8 is a flowchart illustrating a normal distribution test process for continuous variables.

For a continuous variable, a significance probability value (p value; p1) is calculated through execution of Lilliefors test and a significance probability value (p value: p2) is calculated through execution of Shapiro-Wilk test.

The obtained significance probability values p1 and p2 are compared with a reference value (α) and a normal distribution test result for the continuous variable is produced.

According to a normal distribution test result for continuous variables, when the condition p1<α (AND or OR) p2<α is satisfied, it is determined that a normal distribution is not followed, or when the condition is not satisfied, it is determined that the normal distribution is followed.

Herein, the reference value (α) may be set to a value such as 0.05, 0.01, etc., and may be set by a user through the reference value setting means 34a.

After the normal distribution test for the continuous variables is performed, it is determined whether all the variables follow a normal distribution. When the normal distribution is followed, Pearson correlation analysis is set and data analysis control is performed. When the normal distribution is not followed, Spearman correlation analysis is set and data analysis control is performed. An analysis result is provided in the form of an analysis result table and an analysis result figure.

Values provided in an analysis result table as an analysis result for the continuous variable-to-continuous variable relation include:
(a). A correlation coefficient
(b). The 95% confidence interval of a correlation coefficient
(c). A significance probability value (p value) calculated as a result of testing correlation coefficient=0

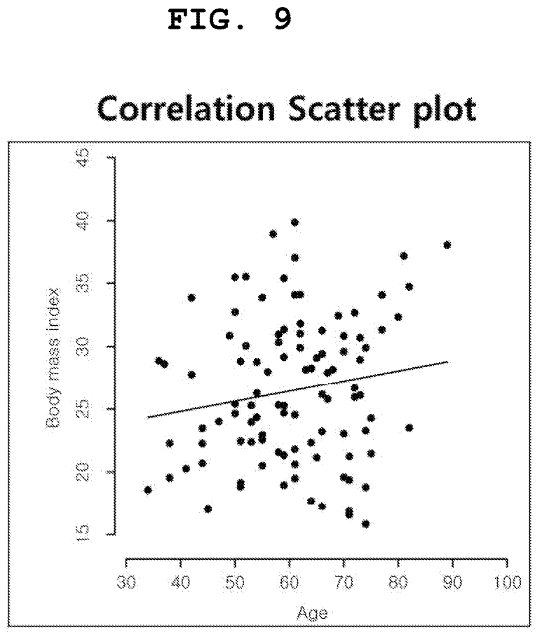

An analysis result figure provided as an analysis result for the continuous variable-to-continuous variable relation may be a correlation scatter plot in which x-axis and y-axis variables are set and a regression curve is represented as shown in FIG. 9.

### [A Continuous Variable-to-Categorical Variable Relation]

In the case of (D). the continuous variable-to-categorical variable relation, a data analysis is performed by distinguishing between a mean difference analysis, a correlation analysis, and a binary response prediction performance analysis.

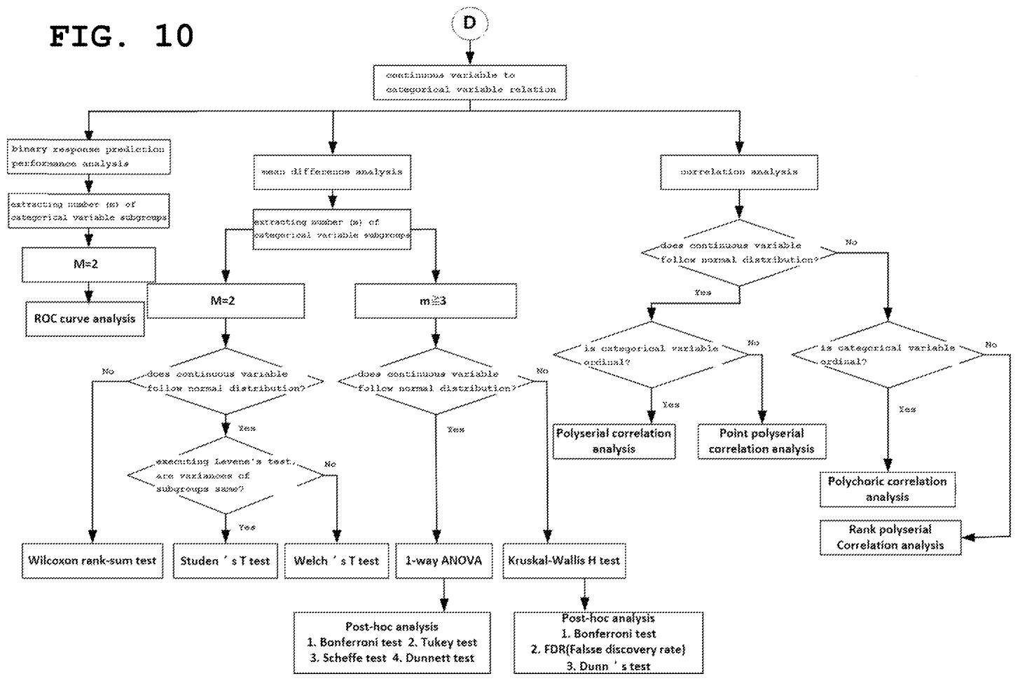

FIG. 10 is a flowchart illustrating a data analysis control process for a continuous variable-to-categorical variable relation.

In the case of the mean difference analysis, the number (m) of categorical variable subgroups is extracted. A normal distribution test, as shown in FIG. 8, for a continuous variable is performed when the extracted number (m) of categorical variable subgroups is two or when the extracted number of categorical variable subgroups is three or more, and according to a result, an algorithm is set.

When the number (m) of categorical variable subgroups is two, it is determined whether a continuous variable follows a normal distribution. When the continuous variable does not follow the normal distribution, Wilcoxon rank-sum test is set. When the continuous variable follows the normal distribution, Levene's test is performed to test whether the variances of the subgroups are the same. When the variances of the subgroups are the same, “Student's Test” is set. When the variances of the subgroups are not the same, “Welch's T test” is set to analyze data.

When the number (m) of categorical variable subgroups is three or more and a continuous variable follows a normal distribution, 1-way ANOVA is set to perform data analysis control. When the continuous variable does not follow the normal distribution, Kruskal-Wallis H test is set to analyze data.

After the data analysis with 1-way ANOVA or Kruskal-Wallis H test, post-validation (post-hoc analysis) is performed.

Bonferroni test, Tukey test, Scheffe test, and Dunnett test are set and performed, thereby performing post-validation after the data analysis with 1-way ANOVA. Bonferroni test, FDR (False Discovery rate), and Dunn's test are set and performed, thereby performing post-validation after the data analysis with Kruskal-Wallis H test.

In the correlation analysis, a normal distribution analysis for a continuous variable is performed as shown in FIG. 8, and it is determined whether the continuous variable follows a normal distribution.

When the continuous variable follows the normal distribution, it is determined whether the categorical variable is ordinal. When the categorical variable is ordinal, Polyserial correlation analysis is set to perform data analysis control. When the categorical variable is not ordinal, Point polyserial correlation analysis is set to perform a data analysis.

When the continuous variable does not follow the normal distribution, it is determined whether the categorical variable is an ordinal variable. When the categorical variable is an ordinal variable, Polychoric correlation analysis is set to perform a data analysis. When the categorical variable is not a nominal variable, Rank polyserial correlation analysis is set to perform a data analysis.

In the binary response prediction performance analysis, the number (m) of categorical variable subgroups is extracted, and when the number (m) of categorical variable subgroups is two, ROC curve analysis algorithm is set to analyze data.

### [A Categorical Variable-to-Categorical Variable Relation]

The case of (E). the categorical variable-to-categorical variable relation will be described.

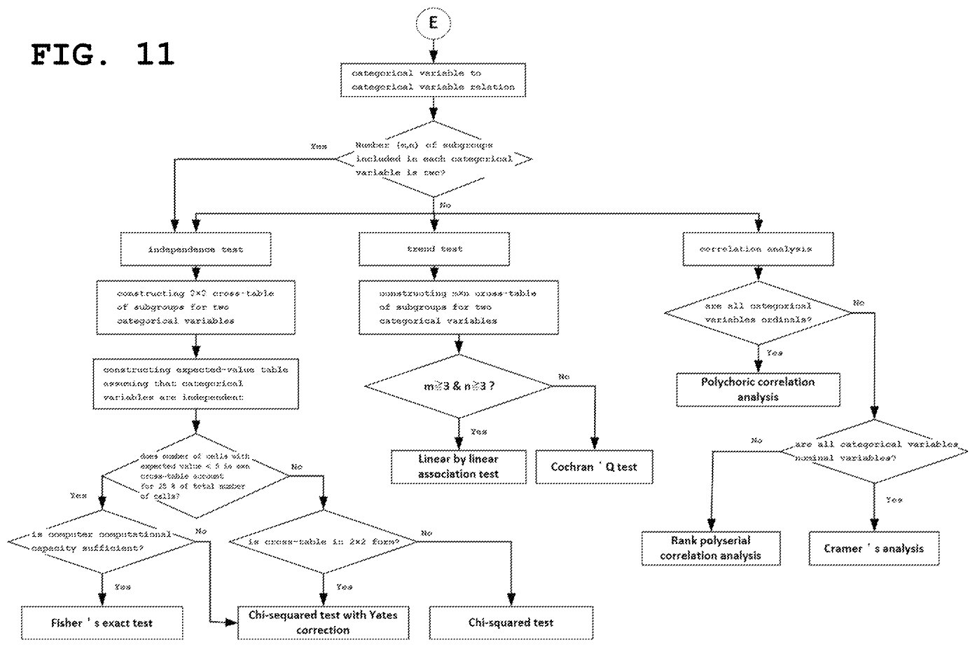

FIG. 11 is a flowchart illustrating an analysis control process for a categorical variable-to-categorical variable relation in the system according to the present disclosure.

A case in which the number (m,n) of subgroups included in each categorical variable is two and other cases are distinguished. When the number (m,n) of subgroups included in each categorical variable is two, a data analysis is performed with an independence test. When the number (m,n) of subgroups included in each categorical variable is not two, a data analysis is performed by distinguishing between an independence test, a trend test, and a correlation analysis.

In the independence test, a 2×2 cross-table of subgroups for two categorical variables is constructed. Assuming that the categorical variables are independent, an expected-value table is constructed. It is determined whether the number of cells with an expected value<5 in an m×n cross-table for the number (m,n) of subgroups included in each categorical variable is equal to or greater than 25% of the total number of cells.

When the number of cells with an expected value<5 in an m×n cross-table for the number (m,n) of subgroups included in each categorical variable is equal to or greater than 25% of the total number of cells, computer computational capacity is considered. When the computer computational capacity is sufficient, Fisher's exact test is set. When the computer computational capacity is insufficient, Chi-squared test with Yates correction is set to perform a data analysis.

When the number of cells with an expected value<5 in the m×n cross-table for the number (m,n) of subgroups included in each categorical variable is less than 25% of the total number of cells, it is determined whether the cross-table is in a 2×2 form. When the cross-table is in a 2×2 form, Chi-squared test with Yates correction is set. When the cross-table is not in a 2×2 form, Chi-squared test is set to perform a data analysis.

This follows the statistical analysis that data has a chi-squared distribution when an expected value is equal to or greater than 25%, or does not follow a chi-squared distribution when an expected value is less than 25%.

For example, two categorical variables, a sex variable and a sweet and sour pork eating style variable, are provided for a sweet and sour pork eating style. In the case of men: dipping style 25, pouring style 25, women: dipping style 25, pouring style 25, this is interpreted as no difference in sweet and sour pork eating style between the sexes. This is statistically analyzed as “sex and sweet and sour pork eating style are independent.”

However, when men: dipping style 10, pouring style 40, women: dipping style 35, pouring style 15 are observed, this may be interpreted as men like pouring style and women like dipping style.

Therefore, there is a relation between sex and sweet and sour pork eating style, so they are “dependent”.

In the trend test, an m×n cross-table of subgroups for two categorical variables is constructed, and it is determined whether the condition m≥3 and n≥3 is satisfied. When the condition m≥3 and n≥3 is satisfied, Linear by linear association test is selected and set. When the condition m≥3 and n≥3 is not satisfied, Cochran'Qtest is set to perform a data analysis.

Herein, since 2×2 only has binary form, such as (0,1) or (Yes,No), “trend” and “independence” have substantially the same meaning, so an analysis is performed only in terms of independence. However, when both m and n are equal to or greater than 3, both the independence test and the trend test may be performed.

The trend test may be explained using an age group variable and an OTP usage type variable (not used, used only one, used two or more, as an example. That is, this may be considered that the “trend”, such as increasing or decreasing, is analyzed. For example, as the age group increases, the number of people using OTT services increases or decreases or does not change as the age group.

In the correlation analysis, it is determined whether all the categorical variables are ordinals. When all the categorical variables are ordinals, Polychoric correlation analysis is selected and set. When not all the categorical variables are ordinals, it is determined whether all the categorical variables are nominal variables. When all the categorical variables are nominal variables, Cramer's V analysis is selected and set. When not all the categorical variables are nominal variables, Rank polyserial correlation analysis is set to perform a data analysis.

[An analysis of a relation between three or more variables that are all single-measured]

For (F). the analysis of a relation between three or more variables that are all single-measured,

a data analysis is performed by distinguishing between the cases in which three or more variable groups are all continuous, are all categorical, and continuous and categorical variables are mixed.

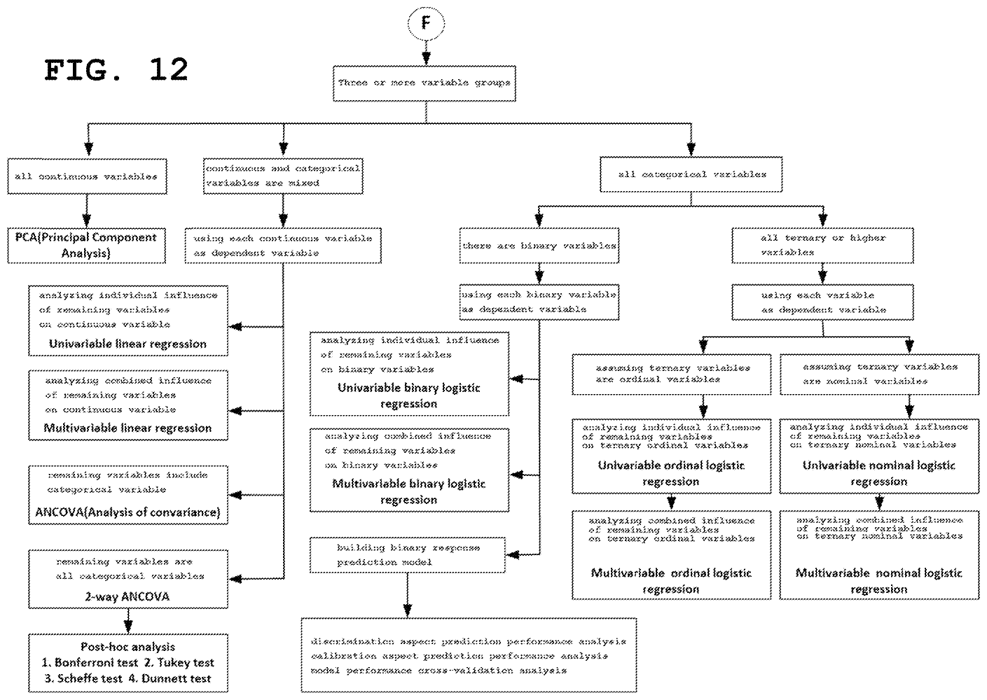

FIG. 12 is a flowchart illustrating a data analysis control process for a relation between three or more variables that are all single-measured in the present disclosure.

When all the variables are continuous, PCA (Principal Component Analysis) is set to perform a data analysis.

When continuous and categorical variables are mixed, the following procedures are performed. Each continuous variable is used as a dependent variable and univariable Linear regression is set to analyze individual influence of the remaining variables on the continuous variable. Each continuous variable is used as a dependent variable and multivariable linear regression is set to analyze combined influence of the remaining variables on the continuous variable. Each continuous variable is used as a dependent variable, and when the remaining variables include a categorical variable, ANCOVA (Analysis of covariance) is set to perform a data analysis. Each continuous variable is used as a dependent variable, and when the remaining variables are all categorical variables, 2-way ANOVA is performed to analyze data.

When all the variables are categorical variables, a data analysis is performed depending on whether binary variables are included. When there are binary variables, each binary variable is used as a dependent variable and individual influence of the remaining variables on the binary variables is analyzed and combined influence of the remaining variables on the binary variables is analyzed.

Univariable binary logistic regression is set to analyze individual influence of the remaining variables on the binary variables. Multivariable binary logistic regression is set to analyze combined influence of the remaining variables on the binary variables.

In addition, a binary response prediction model may be built, and a validation analysis for the built binary response prediction model may be performed.

A validation analysis for the binary response prediction model is performed through a discrimination aspect prediction performance analysis, a calibration aspect prediction performance analysis, and a model performance cross-validation analysis.

[An Analysis when all Variables are Repeatedly Measured]

For (G). the analysis when all variables are repeatedly measured,

a data analysis is performed by distinguishing between the cases in which there are two repeatedly measured variables and there are three or more repeatedly measured variables, and between the cases in which repeatedly measured variables are continuous variables and repeatedly measured variables are categorical variables.

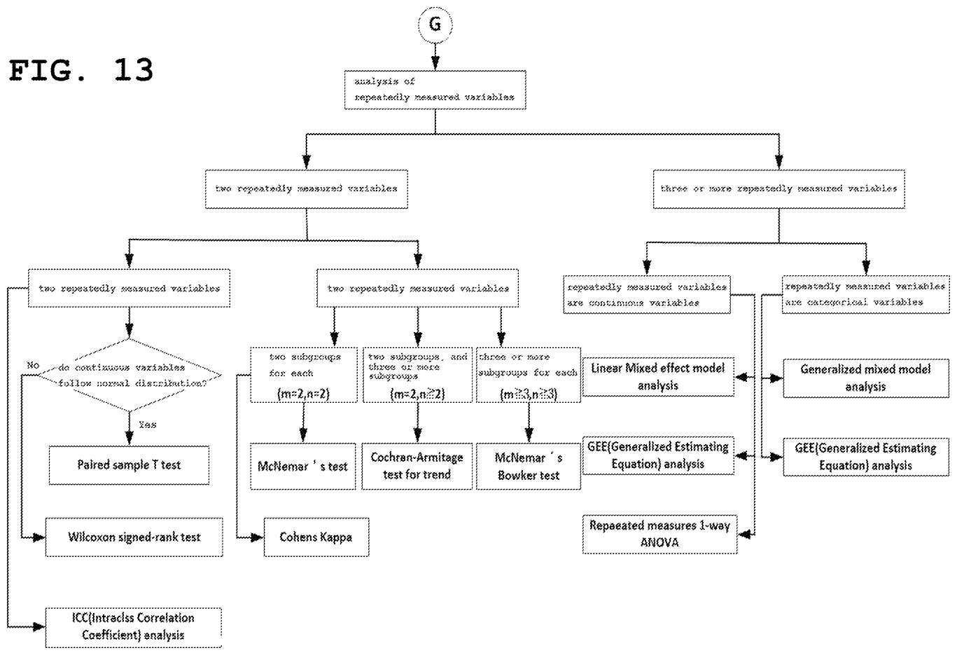

FIG. 13 is a flowchart illustrating a data analysis control process for a case in which all variables are repeatedly measured in the system according to the present disclosure.

When there are two repeatedly measured variables and the repeatedly measured variables are continuous variables, a normal distribution analysis for the continuous variables is performed to determine whether the continuous variables follow a normal distribution. Paired sample T test is set when the normal distribution is followed or Wilcoxon signed-rank test is set when the normal distribution is not followed to analyze data.

In addition, when there are two repeatedly measured variables and the repeatedly measured variables are continuous variables, ICC (Intraclass Correlation Coefficient) analysis is set to analyze data.

When there are two repeatedly measured variables and the repeatedly measured variables are categorical variables, the following procedures are performed. When the number (m,n) of subgroups included in each categorical variable is two (m=2,n=2), McNemar's test and Cohens'Kappa are set to perform a data analysis. When the number (m) of subgroups included in one categorical variable is two and the number (n) of subgroups included in the other is three or more (m=2, n≥3), Cochran-Armitage test for trend is set to analyze data. When the number (m,n) of subgroups included in each categorical variable is three or more (m≥3,n≥3), McNemar-Bowker test is set to analyze data.

When there are three or more repeatedly measured variables and the repeatedly measured variables are continuous variables, Linear mixed effect model analysis, GEE (Generalized Estimating Equation) analysis, and Repeated measures 1-way ANOVA are set to analyze data.

When there are three or more repeatedly measured variables and the repeatedly measured variables are categorical variables, Generalized mixed effect model analysis and GEE (Generalized Estimating Equation) analysis are set to analyze data.

[An Analysis when a Single-Measured Variable is Mixed]

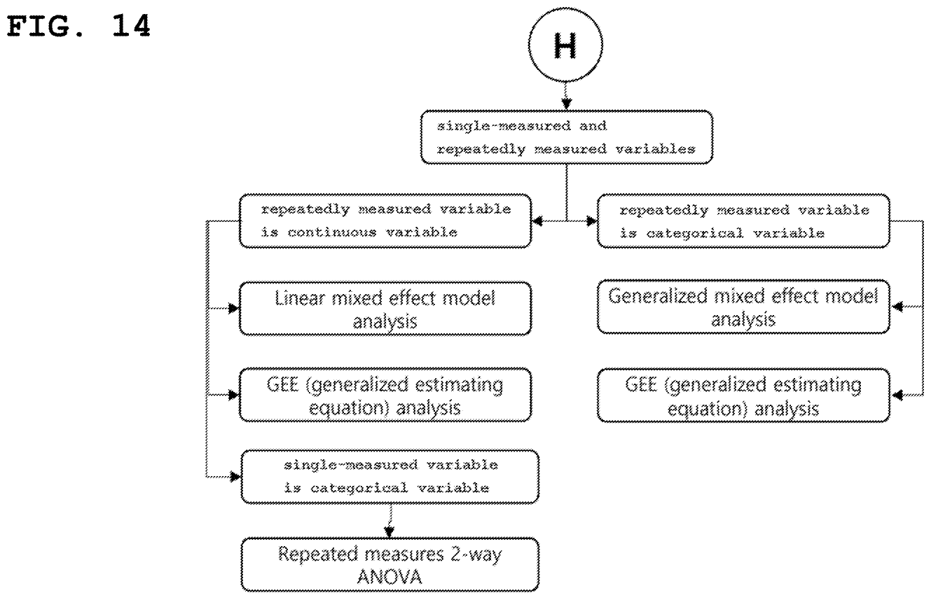

FIG. 14 is a flowchart illustrating a data analysis control process for a case in which a single-measured variable is mixed, in the system according to the present disclosure.

For (H). the analysis when a single-measured variable is mixed, a case in which a repeatedly measured variable is a continuous variable and a case in which a repeatedly measured variable is a categorical variable are distinguished.

When a repeatedly measured variable is a continuous variable, Linear mixed effect model analysis and GEE (Generalized Estimating Equation) analysis are set to perform a data analysis. When a repeatedly measured variable is a continuous variable and a single-measured variable is a categorical variable, Repeated measures 2-way ANOVA is set to perform a data analysis.

When a repeatedly measured variable is a categorical variable, Generalized mixed effect model analysis, and GEE (Generalized Estimating Equation) analysis are set to perform a data analysis.

### [Survival Data Analysis]

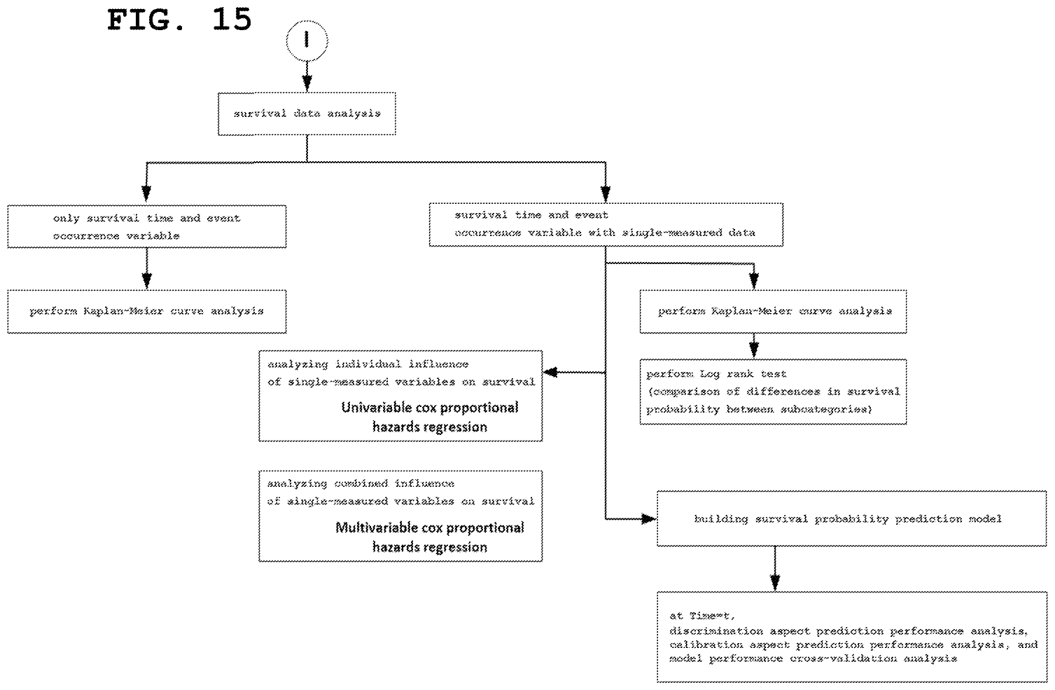

FIG. 15 is a flowchart illustrating a data analysis control process for survival data in the system according to the present disclosure.

For (I). the survival data analysis, a case in which there are only survival time and an event occurrence variable and a case in which there are survival time, an event occurrence variable, and single-measured data are distinguished.

When there are only survival time and an event occurrence variable, Kaplan-Meier curve analysis is set to perform data analysis control. When there are survival time, event occurrence data, and single-measured data, univariable cox proportional hazards regression is set to analyze individual influence of the single-measured variables on survival, and multivariable cox proportional hazards regression is set to analyze combined influence of the single-measured variables on survival.

In addition, Kaplan-Meier curve analysis and Log rank test (comparison of differences in survival probability between subcategories) are performed.

In addition, in a data analysis control process for the survival data,
a survival probability prediction model is built, and a discrimination aspect prediction performance analysis at Time=t, a calibration aspect prediction performance analysis at time=t, and a survival probability prediction model cross-validation analysis are performed to analyze the survival probability prediction model.

In this way, according to the system of the present disclosure, cases required for data analysis are classified according to variable attributes, and according to the classification, an algorithm required for a data analysis is selected and set, and using this, data analysis is automatically performed, thereby providing an automation system that enables a data analysis optimized for a user's desired purpose.

In the meantime, in the system according to the present disclosure, an exploratory data analysis automation method based on variable attributes includes:
a variable attribute definition process of defining variable attributes for statistical analysis automation; and a data analysis process of selecting and setting an algorithm for a data analysis according to the variable attributes and performing the data analysis.

The variable attribute definition process of defining the variable attributes for statistical analysis automation includes:
performing classification into first classification variables and second classification variables, wherein the first classification variables include continuous variables and categorical variables according to forms of constituent values of variables included in data, and the second classification variables include single-measured variables, repeatedly measured variables, and survival data according to features of variables included in data; and defining the attributes.

The categorical variables may be classified into an ordinal variable and a nominal variable.

The continuous variables are variables that represent age, body mass index, height, etc. The ordinal variable is a categorical variable having order, such as an age of 40 or older, or an age younger than 40. The nominal variable is a categorical variable not having order, such as a male/female variable.

The single-measured variables means variables (e.g., a health checkup value measured in 2022) representing values measured one time and independently. The repeatedly measured variables mean variables (e.g., a hemoglobin value measured before surgery/i month after surgery/i year after surgery) representing values measured repeatedly for a predetermined period. The survival data means variables (e.g., survival/censored/death information for 10 years of follow-up) representing sample status and duration information.

In addition, a data analysis process of selecting an algorithm for a data analysis according to the variable attributes and performing the data analysis includes the following processes:

a variable relation analysis process in which a variable relation analysis of variables automatically classified as shown in FIG. 2 is performed according to a combination of the types of the variables and it is determined whether a single-variable distribution analysis or an analysis of a relation between two or more variables is performed,
a single-variable distribution analysis process in which according to a variable relation analysis result of the variable relation analysis process, in the case of the single-variable distribution analysis, distribution for each continuous variable or categorical variable is analyzed and a result is provided as a distribution analysis result table and a distribution analysis result figure, and
according to a variable relation analysis result of the variable relation analysis process,
a variable-to-variable relation analysis process in which in the case of the analysis of a relation between two or more variables, an algorithm is selected and set by distinguishing between the cases of all single-measured variables, all repeatedly measured variables, a mix of single-measured and repeatedly measured variables, and survival data, and a data analysis result is provided.

In addition, the single-variable distribution analysis process includes a normal distribution test for a continuous variable. The normal distribution test as shown in FIG. 8 includes: calculating a significance probability value (p value; p1) through execution of Lilliefors test; calculating a significance probability value (p value: p2) through execution of Shapiro-Wilks test; producing a normal distribution test result for the continuous variable through comparison of a set reference value (α) with p1 and p2.

According to the normal distribution test result for the continuous variable, when the condition p1<α (AND or OR) p2<α is satisfied, it is determined that a normal distribution is not followed, or when the condition is not satisfied, it is determined that the normal distribution is followed.

The set reference value (α) may be a value set by a user.

The single-variable distribution analysis process may provide continuous variable distribution in the form of a distribution analysis result table, a histogram, a Q-Q plot, and a box plot as shown in FIGS. 5a to 5c as a data analysis result, and may provide categorical variable distribution in the form of a distribution analysis result table and a bar plot as shown in FIG. 6 as a data analysis result.

Values calculated from the continuous variable distribution analysis result table include:
(a). Total N the total number of samples included in data
(b). Valid N (%) the number and % of samples excluding missing values
(c). Missing N (%) the number and % of missing samples
(d). Min˜Max the minimum maximum value of the variable
(e). Mean±standard deviation the mean±standard deviation of the variable
(f). Mean (95% CIs) the mean (95% confidence interval) of the variable
(g). Median (IQR) the median (inter-quartile range) of the variable
(h). Skewness the skewness of the variable
(i). Kurtosis the kurtosis of the variable
(j). Lilliefors test for normality, p value
   : a significance probability value (p value) resulting from testing the normal distribution of the variable with the Lilliefors method
(k). Shapiro-Wilk test for normality, p value: a p value resulting from testing the normal distribution of the variable with the Shapiro-Wilk method

For the categorical variable a distribution analysis result table and a bar plot are provided as a data analysis result.

Values calculated from the categorical variable distribution analysis result table include:
(a). Total N: the total number of samples included in data
(b). Valid N (%): the number and % of samples excluding missing values
(c). Missing N (%): the number and % of missing samples
(d). Subgroup: the name of a subgroup included in the categorical variable
(e). N (%): the number and % of samples included in the subgroup
(f). 95% CI: the 95% confidence interval of the number % of samples included in the subgroup

### [When all Variables are Single-Measured]

In the variable-to-variable relation analysis process, when all variables are single-measured, a data analysis is performed by distinguishing between an analysis of a relation between two variables and an analysis of a relation between three or more variables. In the case of the analysis of a relation between two variables, a data analysis is performed according to (C). a continuous variable-to-continuous variable relation, (D). a continuous variable-to-categorical variable relation, and (E). a categorical variable-to-categorical variable relation. In the case of (F). an analysis of a relation between three or more variables, a data analysis result is provided by distinguishing between the cases of all continuous variables, all categorical variables, and a mix of continuous and categorical variables.

### [A Continuous Variable-to-Continuous Variable Relation]

In the case of (C). the continuous variable-to-continuous variable relation, performing a normal distribution test for two continuous variables; and determining whether all the two variables follow a normal distribution as a normal distribution test result, and setting “Pearson correlation analysis” to perform a data analysis when the normal distribution is followed or setting “Spearman correlation analysis” to perform a data analysis when the normal distribution is not followed, and providing an analysis result in the form of an analysis result table and an analysis result figure.

Values provided in an analysis result table as an analysis result for the continuous variable-to-continuous variable relation include:
(a). A correlation coefficient
(b). The 95% confidence interval of a correlation coefficient
(c). A significance probability value (p value) calculated as a result of testing correlation coefficient=0

An analysis result figure provided as an analysis result for the continuous variable-to-continuous variable relation may be a correlation scatter plot in which x-axis and y-axis variables are set and a regression curve is represented as shown in FIG. 9.

### [A Continuous Variable-to-Categorical Variable Relation]

In the case of (D). the continuous variable-to-categorical variable relation,
a data analysis is performed by distinguishing between a mean difference analysis, a correlation analysis, and a binary response prediction performance analysis,

In the case of the mean difference analysis, the following procedures are performed. The number (n) of categorical variable subgroups are extracted, and a normal distribution test for a continuous variable is performed when the extracted number (n) of categorical variable subgroups is two or when the extracted number of categorical variable subgroups is three or more. When the number (n) of categorical variable subgroups is two, it is determined whether the continuous variable follows a normal distribution. When the number (n) of categorical variable subgroups is two and the continuous variable does not follow the normal distribution “Wilcoxon rank-sum test” is set. When the number (n) of categorical variable subgroups is two and the continuous variable follows the normal distribution, “Levene's test” is performed to test whether the variances of the subgroups are the same. When the variances of the subgroups are the same, “Student's Test” is set. When the variances of the subgroups are not the same, “Welch's T test” is set to analyze data.

When the number (m) of categorical variable subgroups is three or more and a continuous variable follows a normal distribution, 1-way ANOVA is set to perform a data analysis. When the continuous variable does not follow the normal distribution, Kruskal-Wallis H test is set to perform a data analysis.

In addition, after the data analysis with 1-way ANOVA or Kruskal-Wallis H test, post-validation (post-hoc analysis) is performed and controlled.

Algorithms used in post-validation performed after the data analysis with the 1-way ANOVA include Bonferroni test, Tukey test, Scheffe test, and Dunnett test. Algorithms used in post-validation after the data analysis with the Kruskal-Wallis H test include Bonferroni test, FDR (False Discovery rate), and Dunn's test.

In addition, the binary response prediction analysis includes: extracting the number (m) of categorical variable subgroups, and setting a ROC curve analysis algorithm to perform data analysis control when the number (m) of categorical variable subgroups is two.

The correlation analysis includes: performing a normal distribution analysis for a continuous variable; determining whether the continuous variable follows a normal distribution; determining whether the categorical variable is an ordinal when the continuous variable follows the normal distribution; setting Polyserial correlation analysis to perform a data analysis when the categorical variable is an ordinal, or setting Point polyserial correlation analysis to perform a data analysis when the categorical variable is not an ordinal; or determining whether the categorical variable is an ordinal variable when the continuous variable does not follow the normal distribution; and setting Polychoric correlation analysis algorithm to perform a data analysis when the categorical variable is an ordinal variable, or setting Rank polyserial correlation analysis algorithm to perform a data analysis when the categorical variable is not an ordinal variable.

### [A Categorical Variable-to-Categorical Variable Relation]

In (E). the categorical variable-to-categorical variable relation, a case in which the number (m,n) of subgroups included in each categorical variable is two and other cases are distinguished. A data analysis is performed with an independence test when the number (m,n) of subgroups included in each categorical variable is two, or is performed through an independence test, a trend test, and a correlation analysis when the number (m,n) of subgroups included in each categorical variable is not two.

The independence test includes: constructing a 2×2 cross-table of subgroups for two categorical variables, constructing an expected-value table assuming that the categorical variables are independent, and determining whether the number of cells with an expected value<5 in an m×n cross-table for the number (m,n) of subgroups included in each categorical variable is equal to or greater than 25% of the total number of cells; and considering computer computational capacity when the number of cells with an expected value<5 in the m×n cross-table for the number (m,n) of subgroups included in each categorical variable is equal to or greater than 25% of the total number of cells, and setting Fisher's exact test when the computer computational capacity is sufficient or setting Chi-squared test with Yates correction when the computer computational capacity is insufficient, or determining whether the cross-table is in a 2×2 form when the number of cells with an expected value<5 in the m×n cross-table for the number (m,n) of subgroups included in each categorical variable is less than 25% of the total number of cells, and setting Chi-squared test with Yates correction when the cross-table is in a 2×2 form or setting Chi-squared test when the cross-table is not in a 2×2 form to perform a data analysis.

The trend test includes: constructing an m×n cross-table of subgroups of two categorical variables and determining whether the condition m≥3 and n≥3 is satisfied; and selecting and setting Linear by linear association test when the condition m≥3 and n≥3 is satisfied or selecting and setting Cochran'Qtest when the condition m≥3 and n≥3 is not satisfied to analyze data.

The correlation analysis includes: determining whether all the categorical variables are ordinals, selecting and setting Polychoric correlation analysis when all the categorical variables are ordinals or determining whether all the categorical variables are nominal variables when not all the categorical variables are ordinals, and selecting and setting Cramer's V analysis when all the categorical variables are nominal variables or setting Rank polyserial correlation analysis when not all the categorical variables are nominal variables to analyze data.

[An Analysis of a Relation Between Three or More Variables that are all Single-Measured]

In (F). the analysis of a relation between three or more variables, a data analysis is performed by distinguishing between the cases in which three or more variable groups are all continuous, are all categorical, and continuous and categorical variables are mixed.

When the three or more variable groups are all continuous, PCA (Principal Component Analysis) is set to perform a data analysis.

When continuous and categorical variables are mixed, the following procedures are performed: using each continuous variable as a dependent variable and setting univariable Linear regression to analyze individual influence of the remaining variables on the continuous variable; using each continuous variable as a dependent variable and setting multivariable linear regression to analyze combined influence of the remaining variables on the continuous variables; and using each continuous variable as a dependent variable and setting ANCOVA (Analysis of covariance) to perform a data analysis when the remaining variables include a categorical variable, and using each continuous variable as a dependent variable and performing 2-way ANOVA to analyze data when the remaining variables are all categorical variables.

When all variables are categorical variables, the following procedures are performed: determining whether there are binary variables; using each binary variable as a dependent variable when there are binary variables and performing univariable binary logistic regression to analyze individual influence of the remaining variables on the binary variables; using each binary variable as a dependent variable and performing multivariable binary logistic regression to analyze combined influence of the remaining variables on the binary variables; building a binary response prediction model; and performing validation analysis control for the built binary response prediction model.

When all variables are categorical variables, the following procedures are performed: determining whether there are binary variables; using each binary variable as a dependent variable when there are binary variables and setting univariable binary logistic regression to analyze individual influence of the remaining variables on the binary variables, and setting multivariable binary logistic regression to analyze combined influence of the remaining variables on the binary variables; building a binary response prediction model; and performing validation analysis control for the built binary response prediction model.

When there are no binary variables and all ternary or higher variables, the following procedures are performed: using each variable as a dependent variable, and assuming the ternary variables are ordinal variables or nominal variables, and analyzing individual influence and combined influence of the remaining variables on the ternary ordinal variables or the ternary nominal variables, wherein univariable ordinal logistic regression is performed to analyze the individual influence of the remaining variables on the ternary ordinal variables, multivariable ordinal logistic regression is performed to analyze the combined influence of the remaining variables on the ternary ordinal variables, univariable nominal logistic is performed to analyze the individual influence of the remaining variables on the ternary nominal variables, and multivariable nominal logistic regression is performed to analyze the combined influence of the remaining variables on the ternary nominal variables.

A validation analysis for the binary response prediction model is performed through a discrimination aspect prediction performance analysis, a calibration aspect prediction performance analysis, and a model performance cross-validation analysis.

Indexes used in the discrimination aspect prediction performance analysis include:
(a). Performance analysis indexes (including the 95% confidence interval)
   AUC (95% CI)
   Sensitivity, Specificity
   PPV (positive predictive value), NPV (negative predictive value)
   ACC (accuracy), MIS (miss-classification rate)
   FPR (False Positive Rate), FNR (False Negative Rate), FDR (False Discovery Rate), FOR (False Omission Rate)
   LR+ (Positive Likelihood Ratio), LR− (Negative Likelihood Ratio), DOR (Diagnostic Odds Ratio)
(b). Visualization of a performance analysis result
   ROC curve

Indexes used in the calibration aspect performance analysis include:
(a). Performance analysis indexes
   AIC (Akaike Information Criterion)
   BIC (Bayes Information Criterion)
   Nagelkerke R2
   Hosmer-Lemeshow test P value
   Brier score
   Spigelhalter Z score with P value
   Linear regression line in Calibration plot
   Intercept, 95% confidence interval, and p value
   Slope, 95% confidence interval, and p value
(b). Visualization of a performance analysis result
   Calibration plot
   Decile plot
   Calibration belt

The model performance cross-validation analysis includes:
(a). Methods used in performance cross-validation
   LOOCV (leave-one-out cross-validation)
   K-fold CV (cross validation)
   Permutation test
   Bootstrapping
(b). Visualization of a cross-validation result
   ROC curve
   Calibration plot
   Decile plot
   Calibration belt
      [An Analysis when all Variables are Repeatedly Measured]

For the case (G). in which all variables are repeatedly measured, a data analysis by distinguishing between the cases in which there are two repeatedly measured variables and there are three or more repeatedly measured variables, and between the cases in which repeatedly measured variables are continuous variables and repeatedly measured variables are categorical variables.

When there are two repeatedly measured variables and the repeatedly measured variables are continuous variables, a normal distribution analysis for the continuous variables is performed to determine whether the continuous variables follow a normal distribution. Paired sample T test is set when the normal distribution is followed or Wilcoxon signed-rank test is set when the normal distribution is not followed to perform a data analysis.

In addition, the data analysis control process may further include, when there are two repeatedly measured variables and the repeatedly measured variables are continuous variables, setting ICC (Intraclass Correlation Coefficient) analysis to perform a data analysis.

When there are two repeatedly measured variables and the repeatedly measured variables are categorical variables, the following procedures are performed. When the number (m,n) of subgroups included in each categorical variable is two (m=2, n=2), McNemar's test and Cohens'Kappa are set to perform a data analysis. When the number (m) of subgroups included in one categorical variable is two and the number (n) of subgroups included in the other is three or more (m=2, n≥3), Cochran-Armitage test for trend is set to analyze data. When the number (m,n) of subgroups included in each categorical variable is three or more (m≥3, n≥3), McNemar-Bowker test is set to analyze data.

When there are three or more repeatedly measured variables and the repeatedly measured variables are continuous variables, Linear mixed effect model analysis, GEE (Generalized Estimating Equation) analysis, and Repeated measures 1-way ANOVA are set to analyze data.

When there are three or more repeatedly measured variables and the repeatedly measured variables are categorical variables, Generalized mixed effect model analysis and GEE (Generalized Estimating Equation) analysis are set to analyze data.

[An Analysis when a Single-Measured Variable is Mixed]

For the case (H). in which a single-measured variable and a repeatedly measured variable are mixed, a case in which a repeatedly measured variable is a continuous variable and a case in which a repeatedly measured variable is a categorical variable are distinguished.

When a repeatedly measured variable is a continuous variable, Linear mixed effect model analysis and GEE (Generalized Estimating Equation) analysis are set to perform a data analysis. When a repeatedly measured variable is a continuous variable and a single-measured variable is a categorical variable, Repeated measures 2-way ANOVA is set to perform a data analysis.

When a repeatedly measured variable is a categorical variable, Generalized mixed effect model analysis, and GEE (Generalized Estimating Equation) analysis are set to perform a data analysis.

### [Survival Data Analysis]

For (I). the survival data analysis, a case in which there are only survival time and an event occurrence variable and a case in which there are survival time, an event occurrence variable, and single-measured data are distinguished.

When there are only survival time and an event occurrence variable, Kaplan-Meier curve analysis algorithm is set to perform a data analysis. When there are survival time, event occurrence data, and single-measured data, univariable cox proportional hazards regression is set to perform an analysis of individual influence of the single-measured variables on survival, multivariable cox proportional hazards regression is set to analyze combined influence of the single-measured variables on survival, and Kaplan-Meier curve analysis is set to perform Log rank (comparison of differences in survival probability between subcategories).

In addition, the following procedures may be further included: building a survival probability prediction model; and performing a discrimination aspect prediction performance analysis at Time=t, a calibration aspect prediction performance analysis at time=t, and a survival probability prediction model cross-validation analysis to analyze the survival probability prediction model.

Indexes used in the discrimination aspect prediction performance analysis at Time=t include, as described above:

Indexes used in the discrimination aspect performance analysis
(a). Performance analysis indexes (including the 95% confidence interval)
   AUC (95% CI)
   Sensitivity, Specificity
   PPV (positive predictive value), NPV (negative predictive value)
   ACC (accuracy), MIS (miss-classification rate)
   FPR (False Positive Rate), FNR (False Negative Rate), FDR (False Discovery Rate), FOR (False Omission Rate)
   LR+ (Positive Likelihood Ratio), LR− (Negative Likelihood Ratio), DOR (Diagnostic Odds Ratio)
(b). Visualization of a performance analysis result
   ROC curve

Indexes used in the calibration aspect performance analysis include:
(a). Performance analysis indexes
   AIC (Akaike Information Criterion)
   BIC (Bayes Information Criterion)
   Nagelkerke R2
   Hosmer-Lemeshow test P value
   Brier score
   Spigelhalter Z score with P value
   Linear regression line in Calibration plot
   Intercept, 95% confidence interval, and p value
   Slope, 95% confidence interval, and p value
(b). Visualization of a performance analysis result
   Calibration plot
   Decile plot
   Calibration belt

The survival probability prediction model performance cross-validation analysis includes:
(a). Methods used in performance cross-validation
   LOOCV (leave-one-out cross-validation)
   K-fold CV (cross validation)
   Permutation test
   Bootstrapping
(b). Visualization of a cross-validation result
   ROC curve
   Calibration plot
   Decile plot
   Calibration belt

### INDUSTRIAL APPLICABILITY

The present disclosure enables a data analysis by selecting an optimal algorithm based on variable attributes, and provides a data analysis automation system adaptive for variable data that are more diversely increased as clinical processes and fields are diversely segmented, the system being a technology that can be widely used in medical, bio, and statistical analysis industries to realize practical and economical values.

1. An exploratory data analysis automation system based on variable attributes, the exploratory data analysis automation system comprising:
a variable attribute definition means (10) for extracting features of variables included in data, and classifying the variables according to forms and features of constituent values of the variables, and defining attributes of the classified variables; an algorithm management means (20) for storing and managing data analysis algorithms and providing the algorithms according to a request of a data analysis control means (30); the data analysis control means (30) for defining attributes of variables included in data through the variable attribute definition means (10), and selecting an algorithm for a data analysis according to the attributes of the variables, and controlling execution of the data analysis of a data analysis means (40); and the data analysis means (40) for performing a data analysis according to an algorithm set by the data analysis control means (30) and providing result information as a distribution analysis result table and a distribution analysis result figure,
wherein the variable attribute definition means (10) is configured to perform classification into first classification variables and second classification variables and define attributes thereof, wherein the first classification variables are classified into continuous variables and categorical variables according to forms of constituent values of variables included in data and the categorical variables are classified into ordinal variables and nominal variables, and the second classification variables include single-measured variables, repeatedly measured variables, and survival data according to features of variables included in data, and
the data analysis control means (30) comprises: a variable relation analysis control means (31) for performing a variable relation analysis of automatically classified variables according to a combination of types of the variables with reference to variable attributes to determine whether a single-variable distribution analysis or an analysis of a relation between two or more variables is performed; a single-variable distribution analysis control means (32) for performing data analysis control for each continuous variable or each categorical variable when it is determined that a single-variable distribution analysis is performed according to a variable relation analysis result of the variable relation analysis control means (31); and a variable-to-variable relation analysis control means (33) for setting an algorithm and performing data analysis control by distinguishing between cases of all single-measured variables, all repeatedly measured variables, a mix of single-measured and repeatedly measured variables, and survival data when it is determined that an analysis of a relation between two or more variables is performed according to a variable relation analysis result of the variable relation analysis control means (31).

2. The exploratory data analysis automation system of claim 1, wherein the data analysis control means (30) further comprises a normal distribution test means (34) for a continuous variable, and the normal distribution test means is configured to calculate a significance probability value (p value; p1) through execution of Lilliefors test for the continuous variable, calculate a significance probability value (p value: p2) through execution of Shapiro-Wilk test, and produce a normal distribution test result for the continuous variable through comparison of a reference value (α) with the p1 and the p2, and
according to the normal distribution test result of the normal distribution test means (34) for the continuous variable, when a condition p1<α (AND or OR) p2<α is satisfied, it is determined that a normal distribution is not followed, or when the condition is not satisfied, it is determined that the normal distribution is followed.

3. The exploratory data analysis automation system of claim 2, wherein the normal distribution test means (34) for a continuous variable further comprises a reference value setting means (34a) for providing a reference value setting process to allow a user to set a reference value (α).

4. The exploratory data analysis automation system of claim 1 or 2, wherein values calculated from the distribution analysis result table for a continuous variable include:
(a). Total N: the total number of samples included in data
(b). Valid N (%): the number and % of samples excluding missing values
(c). Missing N (%): the number and % of missing samples
(d). Min˜Max: a minimum˜maximum value of the variable
(e). Mean±standard deviation: a mean±standard deviation of the variable
(f). Mean (95% CIs): a mean (95% confidence interval) of the variable
(g). Median (IQR): a median (inter-quartile range) of the variable
(h). Skewness: skewness of the variable
(i). Kurtosis: kurtosis of the variable
(j). Lilliefors test for normality, p value
: a significance probability value (p value) resulting from testing a normal distribution of the variable with Lilliefors method
(k). Shapiro-Wilk test for normality, p value a p value resulting from testing a normal distribution of the variable with Shapiro-Wilk method
values calculated from the distribution analysis result table for a categorical variable include:
(a). Total N: the total number of samples included in data
(b). Valid N (%): the number and % of samples excluding missing values
(c). Missing N (%): the number and % of missing samples
(d). Subgroup: a name of a subgroup included in the categorical variable
(e). N (%): the number and % of samples included in the subgroup
(f). 95% CI: 95% confidence interval of the number % of samples included in the subgroup.

5. The exploratory data analysis automation system of claim 1 or 2, wherein the variable-to-variable relation analysis control means (33) is configured to provide a process of setting an algorithm and performing data analysis control by distinguishing between the cases of all single-measured variables, all repeatedly measured variables, a mix of single-measured and repeatedly measured variables, and survival data, and
[when all variables are single-measured]
when all variables are single-measured, a data analysis is performed by distinguishing between an analysis of a relation between two variables and an analysis of a relation between three or more variables, wherein in the case of an analysis of a relation between two variables, a data analysis is performed according to a continuous variable-to-continuous variable relation, a continuous variable-to-categorical variable relation, and a categorical variable-to-categorical variable relation, and in the case of an analysis of a relation between three or more variables, a data analysis is performed by distinguishing between cases of all continuous variables, all categorical variables, and a mix of continuous and categorical variables.

6. The exploratory data analysis automation system of claim 5, wherein
[a continuous variable-to-continuous variable relation]
in the case of the continuous variable-to-continuous variable relation, a data analysis control process comprises: performing a normal distribution test for two continuous variables; and determining whether all the two variables follow a normal distribution as a normal distribution test result, and setting Pearson correlation analysis to perform data analysis control when the normal distribution is followed or setting Spearman correlation analysis to perform data analysis control when the normal distribution is not followed, and providing an analysis result in a form of a distribution analysis result table and an analysis result figure,
[a continuous variable-to-categorical variable relation]
in the case of the continuous variable-to-categorical variable relation, in a data analysis control process,
a data analysis is performed by distinguishing between a mean difference analysis, a correlation analysis, and a binary response prediction performance analysis,
the mean difference analysis comprises: extracting the number (m) of categorical variable subgroups and performing a normal distribution test for a continuous variable when the extracted number (m) of categorical variable subgroups is two or when the extracted number of categorical variable subgroups is three or more; determining whether the continuous variable follows a normal distribution when the number (m) of categorical variable subgroups is two; setting Wilcoxon rank-sum test when the number (m) of categorical variable subgroups is two and the continuous variable does not follow the normal distribution, or performing Levene's test when the number (m) of categorical variable subgroups is two and the continuous variable follows the normal distribution to test whether variances of the subgroups are the same, and setting “Student's Test” when the variances of the subgroups are the same or setting “Welch's T test” when the variances of the subgroups are not the same to perform data analysis control; or setting 1-way ANOVA to perform data analysis control when the number (m) of categorical variable subgroups is three or more and the continuous variable follows a normal distribution, or setting Kruskal-Wallis H test to perform data analysis control when the continuous variable does not follow the normal distribution,
the correlation analysis comprises: performing a normal distribution analysis for a continuous variable; determining whether the continuous variable follows a normal distribution; determining whether a categorical variable is an ordinal when the continuous variable follows the normal distribution; setting Polyserial correlation analysis to perform data analysis control when the categorical variable is an ordinal or setting Point polyserial correlation analysis to perform data analysis control when the categorical variable is not an ordinal; or determining whether the categorical variable is an ordinal variable when the continuous variable does not follow the normal distribution; and setting Polychoric correlation analysis to perform data analysis control when the categorical variable is an ordinal variable or setting Rank polyserial correlation analysis to perform data analysis control when the categorical variable is not an ordinal variable, and
the binary response prediction analysis comprises: extracting the number (m) of categorical variable subgroups; and setting a ROC curve analysis algorithm to perform data analysis control when the extracted number (m) of categorical variable subgroups is two,
[a categorical variable-to-categorical variable relation]
in the case of the categorical variable-to-categorical variable relation, a data analysis control process comprises:
distinguishing between a case in which the number (m,n) of subgroups included in each categorical variable is two and other cases, and performing data analysis control with an independence test when the number (m,n) of subgroups included in each categorical variable is two or by distinguishing between an independence test, a trend test, and a correlation analysis when the number (m,n) of subgroups included in each categorical variable is not two,
the independence test comprises: constructing a 2×2 cross-table of subgroups for two categorical variables, constructing an expected-value table assuming that the categorical variables are independent, and determining whether the number of cells with an expected value<5 in an m×n cross-table for the number (m,n) of subgroups included in each categorical variable is equal to or greater than 25% of the total number of cells; and considering computer computational capacity when the number of cells with an expected value<5 in the m×n cross-table for the number (m,n) of subgroups included in each categorical variable is equal to or greater than 25% of the total number of cells, and setting Fisher's exact test when the computer computational capacity is sufficient or setting Chi-squared test with Yates correction when the computer computational capacity is insufficient, or determining whether the cross-table is in a 2×2 form when the number of cells with an expected value<5 in the m×n cross-table for the number (m,n) of subgroups included in each categorical variable is less than 25% of the total number of cells, and setting Chi-squared test with Yates correction when the cross-table is in a 2×2 form or setting Chi-squared test when the cross-table is not in a 2×2 form to perform data analysis control,
the trend test comprises: constructing an m×n cross-table of subgroups of two categorical variables and determining whether a condition m≥3 and n≥3 is satisfied; and selecting and setting Linear by linear association test when the condition m≥3 and n≥3 is satisfied or setting Cochran'Qtest when the condition m≥3 and n≥3 is not satisfied to perform data analysis control, and
the correlation analysis comprises: determining whether all categorical variables are ordinals, selecting and setting Polychoric correlation analysis when all the categorical variables are ordinals or determining whether all the categorical variables are nominal variables when not all the categorical variables are ordinals, and selecting and setting Cramer's V analysis when all the categorical variables are nominal variables or setting Rank polyserial correlation analysis when not all the categorical variables are nominal variables to perform data analysis control,
[an analysis of a relation between three or more variables that are all single-measured]
for the analysis of a relation between three or more variables that are all single-measured, a data analysis control process
performs a data analysis by distinguishing between the cases in which three or more variable groups are all continuous, are all categorical, and continuous and categorical variables are mixed,
when the three or more variable groups are all continuous, PCA (Principal Component Analysis) is set to perform data analysis control,
when continuous and categorical variables are mixed, the following procedures are performed: using each continuous variable as a dependent variable and setting univariable linear regression to analyze individual influence of the remaining variables on the continuous variable; using each continuous variable as a dependent variable and setting multivariable linear regression to analyze combined influence of the remaining variables on the continuous variable; and using each continuous variable as a dependent variable and setting ANCOVA (Analysis of covariance) to perform data analysis control when the remaining variables include a categorical variable, and using each continuous variable as a dependent variable and performing 2-way ANOVA to perform data analysis control when the remaining variables are all categorical variables,
when all variables are categorical variables, the following procedures are performed: determining whether there are binary variables; using each binary variable as a dependent variable when there are binary variables and setting univariable binary logistic regression to analyze individual influence of the remaining variables on the binary variables; using each binary variable as a dependent variable, and using each binary variable as a dependent variable when the remaining variables are binary, and building a binary response prediction model; and performing validation analysis control for the built binary response prediction model,
when all variables are categorical variables, the following procedures are performed: determining whether there are binary variables; using each binary variable as a dependent variable when there are binary variables and setting univariable binary logistic regression to analyze individual influence of the remaining variables on the binary variables, and setting multivariable binary logistic regression to analyze combined influence of the remaining variables on the binary variables; building a binary response prediction model; and performing validation analysis control for the built binary response prediction model, and
when there are no binary variables and all ternary or higher variables, the following procedures are performed: using each variable as a dependent variable, and assuming the ternary variables are ordinal variables or nominal variables, and analyzing individual influence and combined influence of the remaining variables on the ternary ordinal variables or the ternary nominal variables, wherein univariable ordinal logistic regression is set to analyze the individual influence of the remaining variables on the ternary ordinal variables, multivariable ordinal logistic regression is set to analyze the combined influence of the remaining variables on the ternary ordinal variables, univariable nominal logistic is set to analyze the individual influence of the remaining variables on the ternary nominal variables, and multivariable nominal logistic regression is set to analyze the combined influence of the remaining variables on the ternary nominal variables.

7. The exploratory data analysis automation system of claim 6, wherein values provided in an analysis result table as an analysis result for the continuous variable-to-continuous variable relation include:
(a). a correlation coefficient
(b). 95% confidence interval of a correlation coefficient
(c). a significance probability value (p value) calculated as a result of testing correlation coefficient=0.

8. The exploratory data analysis automation system of claim 6, wherein an analysis result figure provided as an analysis result for the continuous variable-to-continuous variable relation is a correlation scatter plot in which x-axis and y-axis variables are set and a regression curve is represented.

9. The exploratory data analysis automation system of claim 6, wherein in the case of the continuous variable-to-categorical variable relation, the data analysis control process further comprises
performing and controlling post-validation (post-hoc analysis) after the data analysis with the 1-way ANOVA or the Kruskal-Wallis H test, and
algorithms used in post-validation performed after the data analysis with the 1-way ANOVA include Bonferroni test, Tukey test, Scheffe test, and Dunnett test, and algorithms used in post-validation after the data analysis with the Kruskal-Wallis H test include Bonferroni test, FDR (False Discovery rate), and Dunn's test.

10. The exploratory data analysis automation system of claim 6, wherein a validation analysis for the binary response prediction model is performed through a discrimination aspect prediction performance analysis, a calibration aspect prediction performance analysis, and a model performance cross-validation analysis,
indexes used in the discrimination aspect prediction performance analysis include:
(a). performance analysis indexes (including 95% confidence interval)
AUC (95% CI)
Sensitivity, Specificity
PPV (positive predictive value), NPV (negative predictive value)
ACC (accuracy), MIS (miss-classification rate)
FPR (False Positive Rate), FNR (False Negative Rate), FDR (False Discovery Rate), FOR (False Omission Rate)
LR+ (Positive Likelihood Ratio), LR− (Negative Likelihood Ratio), DOR (Diagnostic Odds Ratio)
(b). Visualization of a performance analysis result
ROC curve
indexes used in the calibration aspect performance analysis include:
(a). performance analysis indexes
AIC (Akaike Information Criterion)
BIC (Bayes Information Criterion)
Nagelkerke R2
Hosmer-Lemeshow test P value
Brier score
Spigelhalter Z score with P value
Linear regression line in Calibration plot
Intercept, 95% confidence interval, and p value
Slope, 95% confidence interval, and p value
(b). visualization of a performance analysis result
Calibration plot
Decile plot
Calibration belt the model performance cross-validation analysis includes:
(a). methods used in performance cross-validation
LOOCV (leave-one-out cross-validation)
K-fold CV (cross validation)
Permutation test
Bootstrapping
(b). visualization of a cross-validation result
ROC curve
Calibration plot
Decile plot
Calibration belt.

11. The exploratory data analysis automation system of claim 1 or 2, wherein
[an analysis when all variables are repeatedly measured]
for the analysis when all variables are repeatedly measured, a data analysis control process
performs a data analysis by distinguishing between cases in which there are two repeatedly measured variables and there are three or more repeatedly measured variables, and between cases in which repeatedly measured variables are continuous variables and repeatedly measured variables are categorical variables,
when there are two repeatedly measured variables and the repeatedly measured variables are continuous variables, a normal distribution analysis for the continuous variables is performed to determine whether the continuous variables follow a normal distribution, and Paired sample T test is set when the normal distribution is followed or Wilcoxon signed-rank test is set when the normal distribution is not followed to perform data analysis control,
when there are two repeatedly measured variables and the repeatedly measured variables are categorical variables, the following procedures are performed: when the number (m,n) of subgroups included in each categorical variable is two (m=2, n=2), McNemar's test and Cohens'Kappa are set to perform data analysis control; when the number (m) of subgroups included in one categorical variable is two and the number (n) of subgroups included in the other is three or more (m=2,n≥3), Cochran-Armitage test for trend is set to perform data analysis control; or when the number (m,n) of subgroups included in each categorical variable is three or more (m≥3,n≥3), McNemar-Bowker test is set to perform data analysis control,
when there are three or more repeatedly measured variables and the repeatedly measured variables are continuous variables, Linear mixed effect model analysis, GEE (Generalized Estimating Equation) analysis, and Repeated measures 1-way ANOVA are set to perform data analysis control, and
when there are three or more repeatedly measured variables and the repeatedly measured variables are categorical variables, Generalized mixed effect model analysis and GEE (Generalized Estimating Equation) analysis are set to perform data analysis control.

12. The exploratory data analysis automation system of claim 11, wherein the data analysis control process further comprises, when there are two repeatedly measured variables and the repeatedly measured variables are continuous variables, setting ICC (Intraclass Correlation Coefficient) analysis to perform data analysis control.

13. The exploratory data analysis automation system of claim 1 or 2, wherein
[an analysis when a single-measured variable is mixed]
when a single-measured variable is mixed, in a data analysis control process,
a case in which a repeatedly measured variable is a continuous variable and a case in which a repeatedly measured variable is a categorical variable are distinguished,
when a repeatedly measured variable is a continuous variable, Linear mixed effect model analysis and GEE (Generalized Estimating Equation) analysis are set to perform data analysis control, and when a repeatedly measured variable is a continuous variable and a single-measured variable is a categorical variable, Repeated measures 2-way ANOVA is set to perform data analysis control, and
when a repeatedly measured variable is a categorical variable, Generalized mixed effect model analysis, and GEE (Generalized Estimating Equation) analysis are set to perform data analysis control.

14. The exploratory data analysis automation system of claim 1 or 2, wherein
[survival data analysis]
in a data analysis control process for the survival data, a case in which there are only survival time and an event occurrence variable and a case in which there are survival time, an event occurrence variable, and single-measured data are distinguished, and
when there are only survival time and an event occurrence variable, Kaplan-Meier curve analysis is set to perform data analysis control, and when there are survival time, event occurrence data, and single-measured data, univariable cox proportional hazards regression is set to control an analysis of individual influence of single-measured variables on survival, multivariable cox proportional hazards regression is set to control an analysis of combined influence of the single-measured variables on survival, and Kaplan-Meier curve analysis is set to control Log rank test (comparison of differences in survival probability between subcategories).

15. The exploratory data analysis automation system of claim 14, wherein the data analysis control process for the survival data further comprises:
building a survival probability prediction model; and performing a discrimination aspect prediction performance analysis at Time=t, a calibration aspect prediction performance analysis at time=t, and a survival probability prediction model cross-validation analysis to analyze and control the survival probability prediction model.

16. An exploratory data analysis automation method based on variable attributes, the exploratory data analysis automation method comprising: a variable attribute definition process of defining variable attributes for data analysis automation; and selecting and setting an algorithm for a data analysis according to the variable attributes,
wherein the variable attribute definition process for defining one variable attribute comprises
performing classification into first classification variables and second classification variables and defining attributes thereof, wherein the first classification variables are classified into continuous variables and categorical variables according to forms of constituent values of variables included in data and the categorical variables are classified into ordinal variables and nominal variables, and the second classification variables include single-measured variables, repeatedly measured variables, and survival data according to features of variables included in data, and
a data analysis process of selecting an algorithm for a data analysis according to variable attributes and performing the data analysis comprises the following processes:
a variable relation analysis process in which a variable relation analysis of variables automatically classified is performed according to a combination of types of the variables and it is determined whether a single-variable distribution analysis or an analysis of a relation between two or more variables is performed,
a single-variable distribution analysis process in which according to a variable relation analysis result of the variable relation analysis process, in the case of the single-variable distribution analysis, distribution for each continuous variable or categorical variable is analyzed and a result is provided as a distribution analysis result table and a distribution analysis result figure, and
a variable-to-variable relation analysis process in which according to a variable relation analysis result of the variable relation analysis process, in the case of the analysis of a relation between two or more variables, an algorithm is selected and set by distinguishing between cases of all single-measured variables, all repeatedly measured variables, a mix of single-measured and repeatedly measured variables, and survival data, and a data analysis result is provided.

17. The exploratory data analysis automation method of claim 16, wherein the single-variable distribution analysis process further comprises a normal distribution test for a continuous variable and the normal distribution test comprises: calculating a significance probability value (p value; p1) through execution of Lilliefors test; calculating a significance probability value (p value: p2) through execution of Shapiro-Wilk test; and producing a normal distribution test result for the continuous variable through comparison of a set reference value (α) with the p1 and the p2, and
according to the normal distribution test result for the continuous variable, when a condition p1<α (AND or OR) p2<α is satisfied, it is determined that a normal distribution is not followed, or when the condition is not satisfied, it is determined that the normal distribution is followed.

18. The exploratory data analysis automation method of claim 16 or 17, wherein
in the single-variable distribution analysis process,
values calculated from the distribution analysis result table for a continuous variable include:
(a). Total N: the total number of samples included in data
(b). Valid N (%): the number and % of samples excluding missing values
(c). Missing N (%): the number and % of missing samples
(d). Min˜Max: a minimum˜maximum value of the variable
(e). Mean±standard deviation: a mean±standard deviation of the variable
(f). Mean (95% CIs): a mean (95% confidence interval) of the variable
(g). Median (IQR): a median (inter-quartile range) of the variable
(h). Skewness: skewness of the variable
(i). Kurtosis: kurtosis of the variable
(j). Lilliefors test for normality, p value
: a significance probability value (p value) resulting from testing a normal distribution of the variable with Lilliefors method
(k). Shapiro-Wilk test for normality, p value: a p value resulting from testing a normal distribution of the variable with Shapiro-Wilk method
values calculated from the distribution analysis result table for a categorical variable include:
(a). Total N: the total number of samples included in data
(b). Valid N (%): the number and % of samples excluding missing values
(c). Missing N (%): the number and % of missing samples
(d). Subgroup: a name of a subgroup included in the categorical variable
(e). N (%): the number and % of samples included in the subgroup
(f). 95% CI: 95% confidence interval of the number % of samples included in the subgroup.

19. The exploratory data analysis automation method of claim 16 or 17, wherein
[when all variables are single-measured]
in the variable-to-variable relation analysis process, when all variables are single-measured, a data analysis is performed by distinguishing between an analysis of a relation between two variables and an analysis of a relation between three or more variables, wherein in the case of an analysis of a relation between two variables, a data analysis is performed according to (C). a continuous variable-to-continuous variable relation, (D). a continuous variable-to-categorical variable relation, and (E). a categorical variable-to-categorical variable relation, and in the case of (F). an analysis of a relation between three or more variables, a data analysis result is provided by distinguishing between cases of all continuous variables, all categorical variables, and a mix of continuous and categorical variables.

20. The exploratory data analysis automation method of claim 19, wherein
[a continuous variable-to-continuous variable relation]
in the case of (C). the continuous variable-to-continuous variable relation, the following procedures are performed: performing a normal distribution test for two continuous variables; and determining whether all the two variables follow a normal distribution as a normal distribution test result, and setting “Pearson correlation analysis” to perform a data analysis when the normal distribution is followed or setting “Spearman correlation analysis” to perform a data analysis when the normal distribution is not followed, and providing an analysis result in a form of an analysis result table and an analysis result figure,
[a continuous variable-to-categorical variable relation]
in the case of (D). the continuous variable-to-categorical variable relation,
a data analysis is performed by distinguishing between a mean difference analysis, a correlation analysis, and a binary response prediction performance analysis,
in the case of the mean difference analysis, the following procedures are performed: extracting the number (m) of categorical variable subgroups, and performing a normal distribution test for a continuous variable when the extracted number (m) of categorical variable subgroups is two or when the extracted number of categorical variable subgroups is three or more; determining whether the continuous variable follows a normal distribution when the number (m) of categorical variable subgroups is two; setting “Wilcoxon rank-sum test” when the number (m) of categorical variable subgroups is two and the continuous variable does not follow the normal distribution, or performing “Levene's test” when the number (m) of categorical variable subgroups is two and the continuous variable follows the normal distribution to test whether variances of the subgroups are the same, and setting “Student's Test” when the variances of the subgroups are the same, or setting “Welch's T test” when the variances of the subgroups are not the same to analyze data, or
when the number (m) of categorical variable subgroups is three or more and the continuous variable follows a normal distribution, 1-way ANOVA is set to perform a data analysis, or when the continuous variable does not follow the normal distribution, Kruskal-Wallis H test is set to perform a data analysis,
the correlation analysis comprises: performing a normal distribution analysis for a continuous variable; determining whether the continuous variable follows a normal distribution; determining whether a categorical variable is an ordinal when the continuous variable follows the normal distribution; setting Polyserial correlation analysis to perform a data analysis when the categorical variable is an ordinal, or setting Point polyserial correlation analysis to perform a data analysis when the categorical variable is not an ordinal; or determining whether the categorical variable is an ordinal variable when the continuous variable does not follow the normal distribution; and setting Polychoric correlation analysis algorithm to perform a data analysis when the categorical variable is an ordinal variable, or setting Rank polyserial correlation analysis algorithm to perform a data analysis when the categorical variable is not an ordinal variable, and
the binary response prediction performance analysis comprises: extracting the number (m) of categorical variable subgroups; and setting a ROC curve analysis algorithm to perform data analysis control when the extracted number (m) of categorical variable subgroups is two,
[a categorical variable-to-categorical variable relation]
in (E). the categorical variable-to-categorical variable relation, a case in which the number (m,n) of subgroups included in each categorical variable is two and other cases are distinguished, and a data analysis is performed with an independence test when the number (m,n) of subgroups included in each categorical variable is two, or is performed through an independence test, a trend test, and a correlation analysis when the number (m,n) of subgroups included in each categorical variable is not two,
the independence test comprises: constructing a 2×2 cross-table of subgroups for two categorical variables, constructing an expected-value table assuming that the categorical variables are independent, and determining whether the number of cells with an expected value<5 in an m×n cross-table for the number (m,n) of subgroups included in each categorical variable is equal to or greater than 25% of the total number of cells; and considering computer computational capacity when the number of cells with an expected value<5 in the m×n cross-table for the number (m,n) of subgroups included in each categorical variable is equal to or greater than 25% of the total number of cells, and setting Fisher's exact test when the computer computational capacity is sufficient or setting Chi-squared test with Yates correction when the computer computational capacity is insufficient, or determining whether the cross-table is in a 2×2 form when the number of cells with an expected value<5 in the m×n cross-table for the number (m,n) of subgroups included in each categorical variable is less than 25% of the total number of cells, and setting Chi-squared test with Yates correction when the cross-table is in a 2×2 form or setting Chi-squared test when the cross-table is not in a 2×2 form to perform a data analysis,
the trend test comprises: constructing an m×n cross-table of subgroups of two categorical variables and determining whether a condition m≥3 and n≥3 is satisfied; and selecting and setting Linear by linear association test when the condition m≥3 and n≥3 is satisfied or selecting and setting Cochran'Qtest when the condition m≥3 and n≥3 is not satisfied to analyze data, and
the correlation analysis comprises: determining whether all categorical variables are ordinals, selecting and setting Polychoric correlation analysis when all the categorical variables are ordinals or determining whether all the categorical variables are nominal variables when not all the categorical variables are ordinals, and selecting and setting Cramer's V analysis when all the categorical variables are nominal variables or setting Rank polyserial correlation analysis when not all the categorical variables are nominal variables to analyze data,
[an analysis of a relation between three or more variables that are all single-measured]
in (F). the analysis of a relation between three or more variables, a data analysis is performed by distinguishing between the cases in which three or more variable groups are all continuous, are all categorical, and continuous and categorical variables are mixed,
when the three or more variable groups are all continuous, PCA (Principal Component Analysis) is set to perform a data analysis,
when continuous and categorical variables are mixed, the following procedures are performed: using each continuous variable as a dependent variable and setting univariable Linear regression to analyze individual influence of the remaining variables on the continuous variable; using each continuous variable as a dependent variable and setting multivariable linear regression to analyze combined influence of the remaining variables on the continuous variable; and using each continuous variable as a dependent variable and setting ANCOVA (Analysis of covariance) to perform a data analysis when the remaining variables include a categorical variable, and using each continuous variable as a dependent variable and performing 2-way ANOVA to analyze data when the remaining variables are all categorical variables,
when all variables are categorical variables, the following procedures are performed: determining whether there are binary variables; using each binary variable as a dependent variable when there are binary variables and performing univariable binary logistic regression to analyze individual influence of the remaining variables on the binary variables; using each binary variable as a dependent variable and performing multivariable binary logistic regression to analyze combined influence of the remaining variables on the binary variables; building a binary response prediction model; and performing validation analysis control for the built binary response prediction model,
when there are no binary variables and all ternary or higher variables, the following procedures are performed: using each variable as a dependent variable, and assuming the ternary variables are ordinal variables or nominal variables, and analyzing individual influence and combined influence of the remaining variables on the ternary ordinal variables or the ternary nominal variables, wherein univariable ordinal logistic regression is performed to analyze the individual influence of the remaining variables on the ternary ordinal variables, multivariable ordinal logistic regression is performed to analyze the combined influence of the remaining variables on the ternary ordinal variables, univariable nominal logistic is performed to analyze the individual influence of the remaining variables on the ternary nominal variables, and multivariable nominal logistic regression is performed to analyze the combined influence of the remaining variables on the ternary nominal variables.

21. The exploratory data analysis automation method of claim 20, wherein
values provided in an analysis result table as an analysis result for the continuous variable-to-continuous variable relation include:
(a). a correlation coefficient
(b). 95% confidence interval of a correlation coefficient
(c). a significance probability value (p value) calculated as a result of testing correlation coefficient=0.

22. The exploratory data analysis automation method of claim 20, wherein the continuous variable-to-categorical variable relation analysis further comprises
performing and controlling post-validation (post-hoc analysis) after the data analysis with the 1-way ANOVA or the Kruskal-Wallis H test, and
algorithms used in post-validation performed after the data analysis with the 1-way ANOVA include Bonferroni test, Tukey test, Scheffe test, and Dunnett test, and algorithms used in post-validation after the data analysis with the Kruskal-Wallis H test include Bonferroni test, FDR (False Discovery rate), and Dunn's test.

23. The exploratory data analysis automation method of claim 20, wherein the mean difference analysis further comprises, when the number (m) of categorical variable subgroups is two, setting a ROC curve analysis to perform data analysis control.

24. The exploratory data analysis automation method of claim 20, wherein in the performing of the validation analysis control for the binary response prediction model, a validation analysis for the binary response prediction model is performed through a discrimination aspect prediction performance analysis, a calibration aspect prediction performance analysis, and a model performance cross-validation analysis,
indexes used in the discrimination aspect prediction performance analysis include:
(a). performance analysis indexes (including 95% confidence interval)
AUC (95% CI)
Sensitivity, Specificity
PPV (positive predictive value), NPV (negative predictive value)
ACC (accuracy), MIS (miss-classification rate)
FPR (False Positive Rate), FNR (False Negative Rate), FDR (False Discovery Rate), FOR (False Omission Rate)
LR+ (Positive Likelihood Ratio), LR− (Negative Likelihood Ratio), DOR (Diagnostic Odds Ratio)
(b). visualization of a performance analysis result
ROC curve
indexes used in the calibration aspect performance analysis include:
(a). performance analysis indexes
AIC (Akaike Information Criterion)
BIC (Bayes Information Criterion)
Nagelkerke R2
Hosmer-Lemeshow test P value
Brier score
Spigelhalter Z score with P value
Linear regression line in Calibration plot
Intercept, 95% confidence interval, and p value
Slope, 95% confidence interval, and p value
(b). visualization of a performance analysis result
Calibration plot
Decile plot
Calibration belt
the model performance cross-validation analysis includes:
(a). methods used in performance cross-validation
LOOCV (leave-one-out cross-validation)
K-fold CV (cross validation)
Permutation test
Bootstrapping
(b). visualization of a cross-validation result
ROC curve
Calibration plot
Decile plot
Calibration belt.

25. The exploratory data analysis automation method of claim 16 or 17, wherein
[an analysis when all variables are repeatedly measured]
for the case (G). in which all variables are repeatedly measured, a data analysis is performed by distinguishing between cases in which there are two repeatedly measured variables and there are three or more repeatedly measured variables, and between cases in which repeatedly measured variables are continuous variables and repeatedly measured variables are categorical variables,
when there are two repeatedly measured variables and the repeatedly measured variables are continuous variables, a normal distribution analysis for the continuous variables is performed to determine whether the continuous variables follow a normal distribution, and Paired sample T test is set when the normal distribution is followed or Wilcoxon signed-rank test is set when the normal distribution is not followed to perform a data analysis,
when there are two repeatedly measured variables and the repeatedly measured variables are categorical variables, the following procedures are performed: when the number (m,n) of subgroups included in each categorical variable is two (m=2, n=2), McNemar's test and Cohens'Kappa are set to perform a data analysis; when the number (m) of subgroups included in one categorical variable is two and the number (n) of subgroups included in the other is three or more (m=2, n≥3), Cochran-Armitage test for trend is set to analyze data; or when the number (m,n) of subgroups included in each categorical variable is three or more (m≥3, n≥3), McNemar-Bowker test is set to analyze data,
when there are three or more repeatedly measured variables and the repeatedly measured variables are continuous variables, Linear mixed effect model analysis, GEE (Generalized Estimating Equation) analysis, and Repeated measures 1-way ANOVA are set to analyze data, and
when there are three or more repeatedly measured variables and the repeatedly measured variables are categorical variables, Generalized mixed effect model analysis and GEE (Generalized Estimating Equation) analysis are set to analyze data.

26. The exploratory data analysis automation method of claim 16 or 17, wherein
[an analysis when a single-measured variable is mixed]
for the case (H). in which a single-measured variable and a repeatedly measured variable are mixed, a case in which a repeatedly measured variable is a continuous variable and a case in which a repeatedly measured variable is a categorical variable are distinguished,
when a repeatedly measured variable is a continuous variable, Linear mixed effect model analysis and GEE (Generalized Estimating Equation) analysis are set to perform a data analysis, and when a repeatedly measured variable is a continuous variable and a single-measured variable is a categorical variable, Repeated measures 2-way ANOVA is set to perform a data analysis, and
when a repeatedly measured variable is a categorical variable, Generalized mixed effect model analysis, and GEE (Generalized Estimating Equation) analysis are set to perform a data analysis.

27. The exploratory data analysis automation method of claim 16 or 17, wherein
[survival data analysis]
for (I). the survival data analysis, a case in which there are only survival time and an event occurrence variable and a case in which there are survival time, an event occurrence variable, and single-measured data are distinguished, and
when there are only survival time and an event occurrence variable, Kaplan-Meier curve analysis algorithm is set to perform a data analysis, and when there are survival time, event occurrence data, and single-measured data, univariable cox proportional hazards regression is set to perform an analysis of individual influence of single-measured variables on survival, multivariable cox proportional hazards regression is set to analyze combined influence of the single-measured variables on survival, and Kaplan-Meier curve analysis is set to perform Log rank (comparison of differences in survival probability between subcategories).

28. The exploratory data analysis automation method of claim 27, wherein when there are survival time, event occurrence variable, and single measured data, the following procedures are further comprised: building a survival probability prediction model; and performing a discrimination aspect prediction performance analysis at Time=t, a calibration aspect prediction performance analysis at time=t, and a survival probability prediction model cross-validation analysis to analyze the survival probability prediction model.
